SEQUENCE LISTING (146 sequences)

SEQ ID NO: 1 - protein, 18 aa, Artificial Sequence
  Synthetic
  MISC_FEATURE (1)..(1): Xaa is S, K, D, A, N, M, T, or V
  MISC_FEATURE (2)..(2): Xaa is W, I, Y, F, P, L, V, H, M, A, T, or S
  MISC_FEATURE (3)..(3): Xaa is L, W, I, M, V, F, M, A, or T
  MISC_FEATURE (4)..(4): Xaa is R, C, K, Q, H, P, or C
  MISC_FEATURE (5)..(5): Xaa is D, R, I, E, N, or Y
  MISC_FEATURE (6)..(6): Xaa is I, W, V, L, or F
  MISC_FEATURE (7)..(7): Xaa is W, V, Y, F, P, L, V, H, or D
  MISC_FEATURE (8)..(8): Xaa is D, S, E, N, Y, or S
  MISC_FEATURE (9)..(9): Xaa is W, L, Y, F, P, L, V, or H
  MISC_FEATURE (10)..(10): Xaa is I, V, L, or F
  MISC_FEATURE (11)..(11): Xaa is C, S, A, P, M, H, or T
  MISC_FEATURE (12)..(12): Xaa is E, D, S, Q, Y, or T
  MISC_FEATURE (13)..(13): Xaa is V, F, I, L, A, F, or M
  MISC_FEATURE (14)..(14): Xaa is L, V, I, M, or F
  MISC_FEATURE (15)..(15): Xaa is S, D, A, N, T, Y, or P
  MISC_FEATURE (16)..(16): Xaa is D, E, N, Y, or S
  MISC_FEATURE (17)..(17): Xaa is F, W, Y, L, P, or H
  MISC_FEATURE (18)..(18): Xaa is K, E, R, H, R, P, or Q
XXXXXXXXXXXXXXXXXX

SEQ ID NO: 2 - protein, 18 aa, Artificial Sequence
  Synthetic
  MISC_FEATURE (4)..(4): Xaa is R, G, A, I, L, V, or F
  MISC_FEATURE (5)..(5): Xaa is D, G,I, L, V, F, S, T, E, or Y
  MISC_FEATURE (6)..(6): Xaa is I, A, W, V, L, or F
  MISC_FEATURE (7)..(7): Xaa is W, A, V, Y, F, L, V, H, or D
  MISC_FEATURE (8)..(8): Xaa is D, A, S, E, or Y
  MISC_FEATURE (10)..(10): Xaa isI, A, V, L, or F
  MISC_FEATURE (11)..(11): Xaa is C, S, A, W, M, H, W, R, V, or T
  MISC_FEATURE (13)..(13): Xaa is V, F, I, L, A, or M
  MISC_FEATURE (17)..(17): Xaa is F, W, Y, L, or H
  MISC_FEATURE (18)..(18): Xaa is K, R, H, A, P, or G
SWLXXXXXWXXEXLSDXX

SEQ ID NO: 3 - protein, 18 aa, Artificial Sequence
  Synthetic
SWLRDIWDWICEVLSDFK

SEQ ID NO: 4 - protein, 18 aa, Artificial Sequence
  Synthetic
  MISC_FEATURE (1)..(18): d- isomers
SWLRDIWDWICEVLSDFK

SEQ ID NO: 5 - protein, 19 aa, Artificial Sequence
  Synthetic
GSWLRDIWDWICEVLSDFK

SEQ ID NO: 6 - protein, 20 aa, Artificial Sequence
  Synthetic
SWLRDIWDWICEVLSDFKTW

SEQ ID NO: 7 - protein, 18 aa, Artificial Sequence
  Synthetic
KFDSLVECIWDWIDRLWS

SEQ ID NO: 8 - protein, 18 aa, Artificial Sequence
  Synthetic
  MISC_FEATURE (1)..(18): d- isomer
KWLCRIWSWISDVLDDFE

SEQ ID NO: 9 - protein, 18 aa, Artificial Sequence
  Synthetic
SIWRDWVDLICEFLSDWK

SEQ ID NO: 10 - protein, 18 aa, Artificial Sequence
  Synthetic
DWLRIIWDWVCSVVSDFK

SEQ ID NO: 11 - protein, 18 aa, Artificial Sequence
  Synthetic
SWLRDIWDWISEVLSDFK

SEQ ID NO: 12 - protein, 18 aa, Artificial Sequence
  Synthetic
  MISC_FEATURE (12)..(12): d-isomer
SWLRDIWDWISEVLSDFK

SEQ ID NO: 13 - protein, 18 aa, Artificial Sequence
  Synthetic
SWLRDIWDWICEVLSDFR

SEQ ID NO: 14 - protein, 18 aa, Artificial Sequence
  Synthetic
SYLRDIWDYICEVLSDFK

SEQ ID NO: 15 - protein, 18 aa, Artificial Sequence
  Synthetic
SYLRDIWDYISEVLSDFK

SEQ ID NO: 16 - protein, 18 aa, Artificial Sequence
  Synthetic
SYLREIWDYISEVLSDFR

SEQ ID NO: 17 - protein, 18 aa, Artificial Sequence
  Synthetic
SWLREIWDWICEVLSDFK

SEQ ID NO: 18 - protein, 18 aa, Artificial Sequence
  Synthetic
  MISC_FEATURE (1)..(1): Ac
  MISC_FEATURE (18)..(18): NH2
SWLRDIWDWICEVLSDFK

SEQ ID NO: 19 - protein, 18 aa, Artificial Sequence
  Synthetic
  MISC_FEATURE (1)..(18): d-isomer
  MISC_FEATURE (1)..(1): Ac
  MISC_FEATURE (18)..(18): NH2
SWLRDIWDWICEVLSDFK

SEQ ID NO: 20 - protein, 19 aa, Artificial Sequence
  Synthetic
  MISC_FEATURE (1)..(1): Ac
  MISC_FEATURE (19)..(19): NH2
GSWLRDIWDWICEVLSDFK

SEQ ID NO: 21 - protein, 20 aa, Artificial Sequence
  Synthetic
  MISC_FEATURE (1)..(1): Ac
  MISC_FEATURE (20)..(20): NH2
SWLRDIWDWICEVLSDFKTW

SEQ ID NO: 22 - protein, 18 aa, Artificial Sequence
  Synthetic
  MISC_FEATURE (1)..(18): d-isomer
  MISC_FEATURE (1)..(1): Ac
  MISC_FEATURE (18)..(18): NH2
KFDSLVECIWDWIDRLWS

SEQ ID NO: 23 - protein, 18 aa, Artificial Sequence
  Synthetic
  MISC_FEATURE (1)..(1): Ac
  MISC_FEATURE (18)..(18): NH2
KWLCRIWSWISDVLDDFE

SEQ ID NO: 24 - protein, 18 aa, Artificial Sequence
  Synthetic
  MISC_FEATURE (1)..(1): Ac
  MISC_FEATURE (18)..(18): NH2
SIWRDWVDLICEFLSDWK

SEQ ID NO: 25 - protein, 18 aa, Artificial Sequence
  Synthetic
  MISC_FEATURE (1)..(1): Ac
  MISC_FEATURE (18)..(18): NH2
DWLRIIWDWVCSVVSDFK

SEQ ID NO: 26 - protein, 18 aa, Artificial Sequence
  Synthetic
  MISC_FEATURE (1)..(1): Ac
  MISC_FEATURE (18)..(18): NH2
SWLRDIWDWISEVLSDFK

SEQ ID NO: 27 - protein, 18 aa, Artificial Sequence
  Synthetic
  MISC_FEATURE (1)..(18): d-isomer
  MISC_FEATURE (1)..(1): Ac
  MISC_FEATURE (18)..(18): NH2
SWLRDIWDWISEVLSDFK

SEQ ID NO: 28 - protein, 18 aa, Artificial Sequence
  Synthetic
  MISC_FEATURE (1)..(1): Ac
  MISC_FEATURE (18)..(18): NH2
SWLRDIWDWICEVLSDFR

SEQ ID NO: 29 - protein, 18 aa, Artificial Sequence
  Synthetic
  MISC_FEATURE (1)..(1): Ac
  MISC_FEATURE (18)..(18): NH2
SYLRDIWDYICEVLSDFK

SEQ ID NO: 30 - protein, 18 aa, Artificial Sequence
  Synthetic
  MISC_FEATURE (1)..(1): Ac
  MISC_FEATURE (18)..(18): NH2
SYLRDIWDYISEVLSDFK

SEQ ID NO: 31 - protein, 18 aa, Artificial Sequence
  Synthetic
  MISC_FEATURE (1)..(1): Ac
  MISC_FEATURE (18)..(18): NH2
SYLREIWDYISEVLSDFR

SEQ ID NO: 32 - protein, 18 aa, Artificial Sequence
  Synthetic
  MISC_FEATURE (1)..(1): Ac
  MISC_FEATURE (18)..(18): NH2
SWLREIWDWICEVLSDFK

SEQ ID NO: 33 - protein, 18 aa, Artificial Sequence
  Synthetic
SWLRDIWDWISEVLSDFR

SEQ ID NO: 34 - protein, 18 aa, Artificial Sequence
  Synthetic
SWLRDIWDYISEVLSDFR

SEQ ID NO: 35 - protein, 18 aa, Artificial Sequence
  Synthetic
  MISC_FEATURE (1)..(18): d-isomer
SWLRDIWDYISEVLSDFR

SEQ ID NO: 36 - protein, 18 aa, Artificial Sequence
  Synthetic
SWLRDIWDYICEVLSDFR

SEQ ID NO: 37 - protein, 18 aa, Artificial Sequence
  Synthetic
  MISC_FEATURE (1)..(1): Ac
  MISC_FEATURE (18)..(18): NH2
SWLRDIWDWISEVLSDFR

SEQ ID NO: 38 - protein, 18 aa, Artificial Sequence
  Synthetic
  MISC_FEATURE (1)..(1): Ac
  MISC_FEATURE (18)..(18): NH2
SWLRDIWDYISEVLSDFR

SEQ ID NO: 39 - protein, 18 aa, Artificial Sequence
  Synthetic
  MISC_FEATURE (1)..(18): d-isomer
  MISC_FEATURE (1)..(1): Ac
  MISC_FEATURE (18)..(18): NH2
SWLRDIWDYISEVLSDFR

SEQ ID NO: 40 - protein, 18 aa, Artificial Sequence
  Synthetic
  MISC_FEATURE (1)..(1): Ac
  MISC_FEATURE (18)..(18): NH2
SWLRDIWDYICEVLSDFR

SEQ ID NO: 41 - protein, 18 aa, Artificial Sequence
  Synthetic
AWLRDIWDWICEVLSDFK

SEQ ID NO: 42 - protein, 18 aa, Artificial Sequence
  Synthetic
SALRDIWDWICEVLSDFK

SEQ ID NO: 43 - protein, 18 aa, Artificial Sequence
  Synthetic
SWARDIWDWICEVLSDFK

SEQ ID NO: 44 - protein, 18 aa, Artificial Sequence
  Synthetic
SWLADIWDWICEVLSDFK

SEQ ID NO: 45 - protein, 18 aa, Artificial Sequence
  Synthetic
SWLRAIWDWICEVLSDFK

SEQ ID NO: 46 - protein, 18 aa, Artificial Sequence
  Synthetic
SWLRDAWDWICEVLSDFK

SEQ ID NO: 47 - protein, 18 aa, Artificial Sequence
  Synthetic
SWLRDIADWICEVLSDFK

SEQ ID NO: 48 - protein, 18 aa, Artificial Sequence
  Synthetic
SWLRDIWAWICEVLSDFK

SEQ ID NO: 49 - protein, 18 aa, Artificial Sequence
  Synthetic
SWLRDIWDAICEVLSDFK

SEQ ID NO: 50 - protein, 18 aa, Artificial Sequence
  Synthetic
SWLRDIWDWACEVLSDFK

SEQ ID NO: 51 - protein, 18 aa, Artificial Sequence
  Synthetic
SWLRDIWDWIAEVLSDFK

SEQ ID NO: 52 - protein, 18 aa, Artificial Sequence
  Synthetic
SWLRDIWDWICAVLSDFK

SEQ ID NO: 53 - protein, 18 aa, Artificial Sequence
  Synthetic
SWLRDIWDWICEALSDFK

SEQ ID NO: 54 - protein, 18 aa, Artificial Sequence
  Synthetic
SWLRDIWDWICEVASDFK

SEQ ID NO: 55 - protein, 18 aa, Artificial Sequence
  Synthetic
SWLRDIWDWICEVLADFK

SEQ ID NO: 56 - protein, 18 aa, Artificial Sequence
  Synthetic
SWLRDIWDWICEVLSAFK

SEQ ID NO: 57 - protein, 18 aa, Artificial Sequence
  Synthetic
SWLRDIWDWICEVLSDAK

SEQ ID NO: 58 - protein, 18 aa, Artificial Sequence
  Synthetic
SWLRDIWDWICEVLSDFA

SEQ ID NO: 59 - protein, 19 aa, Artificial Sequence
  Synthetic
  MISC_FEATURE (1)..(1): Ac
SWLRDIWDWICEVLSDFKK

SEQ ID NO: 60 - protein, 19 aa, Artificial Sequence
  Synthetic
  MISC_FEATURE (19)..(19): NH2
KSWLRDIWDWICEVLSDFK

SEQ ID NO: 61 - protein, 19 aa, Artificial Sequence
  Synthetic
  MISC_FEATURE (1)..(1): Ac
  MISC_FEATURE (1)..(1): K(palmitoyl)
  MISC_FEATURE (19)..(19): NH2
KSWLRDIWDWICEVLSDFK

SEQ ID NO: 62 - protein, 19 aa, Artificial Sequence
  Synthetic
  MISC_FEATURE (1)..(1): Ac
  MISC_FEATURE (19)..(19): K(palmitoyl)-NH2
SWLRDIWDWICEVLSDFKK

SEQ ID NO: 63 - protein, 18 aa, Artificial Sequence
  Synthetic
  MISC_FEATURE (1)..(1): Ac
  MISC_FEATURE (18)..(18): amino-PEG12-propionyl-NH2
SWLRDIWDWICEVLSDFK

SEQ ID NO: 64 - protein, 18 aa, Artificial Sequence
  Synthetic
  MISC_FEATURE (1)..(1): Ac-amino-PEG12-propionyl
  MISC_FEATURE (18)..(18): NH2
SWLRDIWDWICEVLSDFK

SEQ ID NO: 65 - protein, 19 aa, Artificial Sequence
  Synthetic
  MISC_FEATURE (1)..(1): Ac
  MISC_FEATURE (19)..(19): (succinimido-propionylaminoethyl-mPEG2K)-NH2
SWLRDIWDWICEVLSDFKC

SEQ ID NO: 66 - protein, 18 aa, Artificial Sequence
  Synthetic
SWLRSIWDWICEVLSDFK

SEQ ID NO: 67 - protein, 18 aa, Artificial Sequence
  Synthetic
SWLRLIWDWICEVLSDFK

SEQ ID NO: 68 - protein, 18 aa, Artificial Sequence
  Synthetic
SWLRFIWDWICEVLSDFK

SEQ ID NO: 69 - protein, 18 aa, Artificial Sequence
  Synthetic
SWLRDIWDWIIEVLSDFK

SEQ ID NO: 70 - protein, 18 aa, Artificial Sequence
  Synthetic
SWLRDIWDWIWEVLSDFK

SEQ ID NO: 71 - protein, 18 aa, Artificial Sequence
  Synthetic
SWLRDIWDWIREVLSDFK

SEQ ID NO: 72 - protein, 18 aa, Artificial Sequence
  Synthetic
SWLRDIWDWIPEVLSDFK

SEQ ID NO: 73 - protein, 11 aa, Artificial Sequence
  Synthetic
SWLRDIWDWIR

SEQ ID NO: 74 - protein, 18 aa, Artificial Sequence
  Synthetic
SIWRDIWDLAVEAVSDWK

SEQ ID NO: 75 - protein, 18 aa, Artificial Sequence
  Synthetic
SILRDIWDWICEVLSDFK

SEQ ID NO: 76 - protein, 18 aa, Artificial Sequence
  Synthetic
SLWRDIWDWICEVLSDFK

SEQ ID NO: 77 - protein, 18 aa, Artificial Sequence
  Synthetic
SWLRDIWDLICEVLSDFK

SEQ ID NO: 78 - protein, 18 aa, Artificial Sequence
  Synthetic
SWLRDIWDWIVEVLSDFK

SEQ ID NO: 79 - protein, 18 aa, Artificial Sequence
  Synthetic
SWLRDIWDWICEAVSDFK

SEQ ID NO: 80 - protein, 18 aa, Artificial Sequence
  Synthetic
SWLRDIWDWICEVLSDWK

SEQ ID NO: 81 - protein, 18 aa, Artificial Sequence
  Synthetic
SWLQRIWQWISDVLSEFE

SEQ ID NO: 82 - protein, 18 aa, Artificial Sequence
  Synthetic
SWLQDIWDWICEVLSDFK

SEQ ID NO: 83 - protein, 18 aa, Artificial Sequence
  Synthetic
SWLRRIWDWICEVLSDFK

SEQ ID NO: 84 - protein, 18 aa, Artificial Sequence
  Synthetic
SWLRDIWQWICEVLSDFK

SEQ ID NO: 85 - protein, 18 aa, Artificial Sequence
  Synthetic
SWLRDIWDWICDVLSDFK

SEQ ID NO: 86 - protein, 18 aa, Artificial Sequence
  Synthetic
SWLRDIWDWICEVLSEFK

SEQ ID NO: 87 - protein, 18 aa, Artificial Sequence
  Synthetic
SWLRDIWDWICEVLSDFE

SEQ ID NO: 88 - protein, 18 aa, Artificial Sequence
  Synthetic
SWLRAIWDWISEVLSDFR

SEQ ID NO: 89 - protein, 18 aa, Artificial Sequence
  Synthetic
SWLRAIWDWISEVLSDFR

SEQ ID NO: 90 - protein, 18 aa, Artificial Sequence
  Synthetic
SWLRAIWDWISEVLSDFR

SEQ ID NO: 91 - protein, 18 aa, Artificial Sequence
  Synthetic
SWLRAIWDWISEVLSDFR

SEQ ID NO: 92 - protein, 19 aa, Artificial Sequence
  Synthetic
  MISC_FEATURE (1)..(1): Ac
  MISC_FEATURE (19)..(19): K(oleoyl)-NH2
SWLRDIWDWICEVLSDFKK

SEQ ID NO: 93 - protein, 19 aa, Artificial Sequence
  Synthetic
  MISC_FEATURE (1)..(1): Ac-K(succinyl-D-a-tocopherol)
  MISC_FEATURE (19)..(19): NH2
KSWLRDIWDWICEVLSDFK

SEQ ID NO: 94 - protein, 19 aa, Artificial Sequence
  Synthetic
  MISC_FEATURE (1)..(1): Ac
  MISC_FEATURE (19)..(19): K(succinyl-D-a-tocopherol)-NH2
SWLRDIWDWICEVLSDFKK

SEQ ID NO: 95 - protein, 19 aa, Artificial Sequence
  Synthetic
  MISC_FEATURE (19)..(19): K(palmitoyl)-NH2
SWLRDIWDWICEVLSDFKK

SEQ ID NO: 96 - protein, 19 aa, Artificial Sequence
  Synthetic
  MISC_FEATURE (1)..(1): Ac-C(succinimido-propionylaminoethyl-mPEG2K)-
  MISC_FEATURE (19)..(19): NH2
CSWLRDIWDWICEVLSDFK

SEQ ID NO: 97 - protein, 19 aa, Artificial Sequence
  Synthetic
  MISC_FEATURE (19)..(19): C(cholesteryloxycarbonylmethyl)-NH2
SWLRDIWDWICEVLSDFKC

SEQ ID NO: 98 - protein, 18 aa, Artificial Sequence
  Synthetic
SWLRDIADWIAEVLSDFA

SEQ ID NO: 99 - protein, 18 aa, Artificial Sequence
  Synthetic
SWLRAIADWIAEVLSDFK

SEQ ID NO: 100 - protein, 18 aa, Artificial Sequence
  Synthetic
  MISC_FEATURE (1)..(1): Ac
  MISC_FEATURE (18)..(18): NH2
SWLRDIADWIAEVLSDFA

SEQ ID NO: 101 - protein, 18 aa, Artificial Sequence
  Synthetic
  MISC_FEATURE (1)..(1): Ac
  MISC_FEATURE (18)..(18): NH2
SWLRAIADWIAEVLSDFK

SEQ ID NO: 102 - protein, 18 aa, Artificial Sequence
  Synthetic
  MISC_FEATURE (1)..(1): Ac
  MISC_FEATURE (18)..(18): NH2
SWLRDIADWICEVLSDFK

SEQ ID NO: 103 - protein, 18 aa, Artificial Sequence
  Synthetic
  MISC_FEATURE (1)..(1): Ac
  MISC_FEATURE (18)..(18): NH2
SWLRAIWDWISEVLSDFR

SEQ ID NO: 104 - protein, 18 aa, Artificial Sequence
  Synthetic
SWLRDIWDWIGEVLSDFK

SEQ ID NO: 105 - protein, 18 aa, Artificial Sequence
  Synthetic
SWLRDIADWIAEVLSDFK

SEQ ID NO: 106 - protein, 18 aa, Artificial Sequence
  Synthetic
SWLRDIGDWIAEVLSDFK

SEQ ID NO: 107 - protein, 18 aa, Artificial Sequence
  Synthetic
SWLRDIADWIAEVLSDFR

SEQ ID NO: 108 - protein, 18 aa, Artificial Sequence
  Synthetic
SWLRDIADWICEVLSDWK

SEQ ID NO: 109 - protein, 18 aa, Artificial Sequence
  Synthetic
SWLRDIADWICEALSDFK

SEQ ID NO: 110 - protein, 18 aa, Artificial Sequence
  Synthetic
  MISC_FEATURE (1)..(1): Conj
  MISC_FEATURE (18)..(18): NH2
SWLRDIWDWICEVLSDFK

SEQ ID NO: 111 - protein, 18 aa, Artificial Sequence
  Synthetic
  MISC_FEATURE (1)..(1): Ac
  MISC_FEATURE (18)..(18): Conj
SWLRDIWDWICEVLSDFK

SEQ ID NO: 112 - protein, 18 aa, Artificial Sequence
  Synthetic
  MISC_FEATURE (1)..(1): Conj
  MISC_FEATURE (18)..(18): Conj
SWLRDIWDWICEVLSDFK

SEQ ID NO: 113 - protein, 38 aa, Artificial Sequence
  Synthetic
  MISC_FEATURE (1)..(1): Ac
  MISC_FEATURE (38)..(38): NH2
SWLRDIWDWICEVLSDFKKKSWLRDIWDWICEVLSDFK

SEQ ID NO: 114 - protein, 38 aa, Artificial Sequence
  Synthetic
  MISC_FEATURE (1)..(1): Ac
  MISC_FEATURE (38)..(38): NH2
SWLRDIWDWICEVLSDFKSSSWLRDIWDWICEVLSDFK

SEQ ID NO: 115 - protein, 38 aa, Artificial Sequence
  Synthetic
  MISC_FEATURE (1)..(1): Ac
  MISC_FEATURE (38)..(38): NH2
SWLRDIWDWICEVLSDFKCCSWLRDIWDWICEVLSDFK

SEQ ID NO: 116 - protein, 6 aa, Artificial Sequence
  Synthetic
GSGSGC

SEQ ID NO: 117 - protein, 16 aa, Artificial Sequence
  Synthetic
RKSIHIGPGRAFYTTG

SEQ ID NO: 118 - protein, 16 aa, Artificial Sequence
  Synthetic
RKGIRIGPGRAVYAAE

SEQ ID NO: 119 - protein, 5 aa, Artificial Sequence
  Synthetic
TKPKG

SEQ ID NO: 120 - protein, 4 aa, Artificial Sequence
  Synthetic
TKPR

SEQ ID NO: 121 - protein, 4 aa, Artificial Sequence
  Synthetic
TKPK

SEQ ID NO: 122 - protein, 18 aa, Artificial Sequence
  Synthetic
  misc_feature (1)..(14): Xaa can be any naturally occurring amino acid
XXXXXXXXXXXXXXTKPR

SEQ ID NO: 123 - protein, 18 aa, Artificial Sequence
  Synthetic
  misc_feature (1)..(10): Xaa can be any naturally occurring amino acid
  misc_feature (15)..(18): Xaa can be any naturally occurring amino acid
XXXXXXXXXXTKPRXXXX

SEQ ID NO: 124 - protein, 18 aa, Artificial Sequence
  Synthetic
  misc_feature (5)..(18): Xaa can be any naturally occurring amino acid
TKPRXXXXXXXXXXXXXX

SEQ ID NO: 125 - protein, 13 aa, Artificial Sequence
  Synthetic
GRKKRRQRRRPPQ

SEQ ID NO: 126 - protein, 9 aa, Artificial Sequence
  Synthetic
RKKRRQRRR

SEQ ID NO: 127 - protein, 17 aa, Artificial Sequence
  Synthetic
TRQARRNRRRRWRERQR

SEQ ID NO: 128 - protein, 15 aa, Artificial Sequence
  Synthetic
RRRRNRTRRNRRRVR

SEQ ID NO: 129 - protein, 16 aa, Artificial Sequence
  Synthetic
RQIKIWFQNRRMKWKK

SEQ ID NO: 130 - protein, 16 aa, Artificial Sequence
  Synthetic
RKKRRRESRKKRRRES

SEQ ID NO: 131 - protein, 20 aa, Artificial Sequence
  Synthetic
GLWRALWRLLRSLWRLLWRA

SEQ ID NO: 132 - protein, 17 aa, Artificial Sequence
  Synthetic
KLALKLALKALKAALKA

SEQ ID NO: 133 - protein, 33 aa, Artificial Sequence
  Synthetic
GDCLPHLKLCKENKDCCSKKCKRRGTNIEKRCR

SEQ ID NO: 134 - protein, 18 aa, Artificial Sequence
  Synthetic
LLIILRRRIRKQAHAHSK

SEQ ID NO: 135 - protein, 21 aa, Artificial Sequence
  Synthetic
AGYLLGKINLKALAALAKKIL

SEQ ID NO: 136 - protein, 12 aa, Artificial Sequence
  Synthetic
PLIYLRLLRGQF

SEQ ID NO: 137 - protein, 21 aa, Artificial Sequence
  Synthetic
KETWWETWWTEWSQPKKKRKV

SEQ ID NO: 138 - protein, 15 aa, Artificial Sequence
  Synthetic
SDLWEMMMVSLACQY

SEQ ID NO: 139 - protein, 28 aa, Artificial Sequence
  Synthetic
LSTAADMQGVVTDGMASGLDKDYLKPDD

SEQ ID NO: 140 - protein, 27 aa, Artificial Sequence
  Synthetic
GWTLNSAGYLLGKINLKALAALAKKIL

SEQ ID NO: 141 - protein, 17 aa, Artificial Sequence
  Synthetic
CSIPPEVKFNKPFVYLI

SEQ ID NO: 142 - protein, 12 aa, Artificial Sequence
  Synthetic
AAVLLPVLLAAP

SEQ ID NO: 143 - DNA, 113 bp, Artificial Sequence
  Synthetic
acaggtacgttaatagttaatagcgtacttctttttcttgctttcgtggtattcttgctagttacactagccatccttactgcgcttcgattgtgtgcgtactgctgcaatat

SEQ ID NO: 144 - DNA, 91 bp, Artificial Sequence
  Synthetic
aatcagcacctttaattgaattgtgcgtggatgaggctggttctaaatcacccattcagtacatcgatatcggtaattatacagtttcctg

SEQ ID NO: 145 - DNA, 19 bp, Artificial Sequence
  Synthetic
agatttggacctgcgagcg

SEQ ID NO: 146 - DNA, 20 bp, Artificial Sequence
  Synthetic
gagcggctgtctccacaagt

(19) United States
(12) Patent Application Publication
(10) Pub. No.: US 2024/0197814 A1
(43) Pub. Date: Jun. 20, 2024

(54) MATERIALS AND METHODS FOR THE PREVENTION AND TREATMENT OF VIRAL DISEASES

(71) Applicant: OAK CREST INSTITUTE OF SCIENCE, Monrovia, CA (US)

(72) Inventors: Marc M. Baum, Altadena, CA (US); Manjula Gunawardana, Claremont, CA (US)

(21) Appl. No.: 18/287,202

(22) PCT Filed: Apr. 27, 2022
(86) PCT No.: PCT/US22/26539

§ 371 Date: Oct. 17, 2023

Related U.S. Application Data
(60) Provisional application No. 63/225,261, filed on Jul. 23, 2021.
(60) Provisional application No. 63/180,416, filed on Apr. 27, 2021.

(51) Int. Cl. A61K 38/10; A61K 9/00; A61K 31/675; A61K 45/06; A61P 31/18

(52) U.S. Cl. CPC A61K 38/10; A61K 9/0034; A61K 31/675; A61K 45/06; A61P 31/18

(57) ABSTRACT

This disclosure is related to the use of a peptide with antimicrobial properties. The peptide is administered to prevent or treat non-respiratory diseases caused by viruses. A variety of formulations and uses are described as well as methods of manufacture thereof. The formulations are safe and useful in patients—both humans and animals—for the delivery of appropriate bioactive substance(s).

### FIELD OF INVENTION

This invention is generally in the field of delivery of peptides to treat or prevent disease or disorders.

### BACKGROUND

The global threat posed by existing and emerging viral epidemics represents one of the most critical global public health concerns. While the field of viral therapeutics has advanced in response to this pressing demand, new safe and effective antiviral agents for the prevention and treatment of viral infections are needed urgently.

Of particular concern are enveloped viruses. An enveloped virus is made up of a nucleoprotein core, surrounded by a lipoprotein envelope having a closed lipid bilayer. The lipid is derived from the host cell's membrane(s), with glycoprotein on the outside and matrix protein or nucleocapsid protein on the inside. Exemplary enveloped virus families include Togaviridae, Flaviviridae, Coronaviridae, Rhabdoviridae, Filoviridae, Paramyxoviridae, Orthomyxoviridae, Bunyaviridae, Arenaviridae, Retroviridae, Herpesviridae, Poxviridae and some members of the Iridoviridae. These viruses are responsible for a range of diseases, including: encephalitis, intestinal infections, immunosuppressive disease, respiratory disease, hepatitis, and pox infections. Examples of enveloped viruses include: Human immunodeficiency virus (HIV), herpes simplex viruses (HSV), hepatitis B virus (HBV), hepatitis C virus (HCV), Dengue virus, West Nile virus, measles virus, and Ebola virus, among others.

Non-enveloped viruses also represent a significant concern. For example, the Picornaviridae family include polioviruses, hepatitis A virus, and coxsackieviruses that may cause hand, foot, and mouth disease (HFMD), as well as disease of muscles, lungs, and heart. Papillomaviridae is a family of enveloped viruses that contains over 100 human papillomaviruses (HPV), the most common sexually transmitted infection.

Most antiviral agents are specific to one pathogen, making the development of a drug stockpile for the prevention and treatment of emerging new viruses a significant challenge. There is also a need to prevent and/or treat multiple, intersecting viral pathogens, such as sexually transmitted infection that can comprise HIV, HSV, and HPV. In this regard, new classes of antiviral agents, to be used alone or in combination with existing antiviral agents and therapeutics, are needed. Ideally, the new agent(s) would meet one or more of the following criteria: be safe locally and systemically; act as early as possible in the viral life cycle; be as virus-specific as possible (i.e., attack a target specific to the virus but not the host); render the intact virus noninfectious; prevent the death or dysfunction of virus-infected cells; prevent further production of virus from infected cells; prevent spread of virus infection to uninfected cells; be potent and active against the broadest possible range of strains and isolates of a given virus; be efficacious against emerging new strains (i.e., the agent should not lead to viral resistance); be resistant to degradation under physiological and rigorous environmental conditions; and/or be readily and inexpensively produced.

In view of the foregoing, there is a need in the art for new methods and compositions for inhibiting viral infection. The disclosure provides such methods and compositions. These and other advantages, as well as additional inventive features, will become apparent from the description provided herein.

### SUMMARY

Provided herein are methods of treating or preventing a viral non-respiratory disease, the methods comprising administering to a subject in need thereof a peptide comprising X₁-X₂-X₃-X₄-X₅-X₆-X₇-X₈-X₉-X₁₀-X₁₁-X₁₂-X₁₃-X₁₄-X₁₅-X₁₆-X₁₇-X₁₈ (Formula 1; SEQ ID NO: 1), wherein X₁ is S, K, D, A, N, M, T, or V; X₂ is W, I, Y, F, P, L, V, H, M, A, T, or S; X₃ is L, W, I, M, V, F, M, A, or T; X₄ is R, C, K, Q, H, P, or C; X₅ is D, R, I, E, N, or Y; X₆ is I, W, V, L, or F; X₇ is W, V, Y, F, P, L, V, H, or D; X₈ is D, E, N, Y, or S; X₉ is W, L, Y, F, P, L, V, or H; X₁₀ is I, V, L, or F; X₁₁ is C, S, A, P, M, H, or T; X₁₂ is E, D, S, Q, Y, or T; X₁₃ is V, F, I, L, A, or M; X₁₄ is L, V, I, M, or F; X₁₅ is S, D, A, N, T, Y, or P; X₁₆ is D, E, N, Y, or S; X₁₇ is F, W, Y, L, P, or H; and X₁₈ is K, E, H, R, P, or Q. In some embodiments, the peptide comprises the amino acid sequence of Peptide 346-001. In some embodiments, the peptide comprises the amino acid sequence of Peptide 346-001 having one, two, or three amino acid substitutions, the substitutions being selected from the following: the S at position 1 is substituted with K, D, A, N, M, T, or V; the W at position 2 is substituted with I, Y, F, P, L, V, H, M, A, T, or S; the L at position 3 is substituted with W, I, M, V, F, M, A, or T; the R at position 4 is substituted with C, K, Q, H, P, or C; the D at position 5 is substituted with R, I, E, N, or Y; the I at position 6 is substituted with W, V, L, or F; the W at position 7 is substituted with V, Y, F, P, L, V, H, or D; the D at position 8 is substituted with E, N, Y, or S; the W at position 9 is substituted with L, Y, F, P, L, V, or H; the I at position 10 is substituted with V, L, or F; the C at position 11 is substituted with S, A, P, M, H, or T; the E at position 12 is substituted with D, S, Q, Y, or T; the V at position 13 is substituted with F, I, L, A, or M; the L at position 14 is substituted with V, I, M, or F; the S at position 15 is substituted with D, A, N, T, Y, or P; the D at position 16 is substituted with E, N, Y, or S; the F at position 17 is substituted with W, Y, L, P, or H; or the K at position 18 is substituted with E, H, R, P, or Q.

Also provided are peptides comprising S-W-L-X₄-X₅-X₆-X₇-X₈-W-X₁₀-X₁₁-E-X₁₃-L-S-D-X₁₇-X₁₈ (Formula 2; SEQ ID NO: 2), wherein

X₄ is R, G, A, I, L, V, or F; X₅ is D, G, I, L, V, F, S, T, E, or Y; X₆ is I, A, W, V, L, or F; X₇ is W, A, V, Y, F, L, V, H, or D; X₈ is D, A, S, E, or Y; X₁₀ is I, A, V, L, or F; X₁₁ is C, S, A, W, M, H, W, R, V, or T; X₁₃ is V, F, I, L, A, or M; X₁₇ is F, W, Y, L, or H; and X₁₈ is K, R, H, A, P, or G. Also provided are compositions comprising the a peptide of Formula (2), and methods of treating or preventing a viral non-respiratory disease, the method comprising administering to a subject in need thereof a peptide of Formula (2) or a composition comprising a peptide of Formula (2).

### DESCRIPTION OF THE FIGURES

FIG. 1 illustrates exemplary embodiments of peptide conjugates based on the peptide 346-001 backbone. Conj, denotes conjugate.

FIGS. 2A-C illustrate exemplary embodiments of peptide N-conjugates via a lysine linker based on the peptide 346-001 backbone (SEQ ID NO: 113).

FIGS. 3A-C illustrate exemplary embodiments of peptide O-conjugates via a serine linker based on the peptide 346-001 backbone (SEQ ID NO: 114).

FIGS. 4A-G illustrate exemplary embodiments of peptide S-conjugates via a cysteine linker based on the peptide 346-001 backbone (SEQ ID NO: 115).

FIGS. 5A-C illustrate the results of an in vitro SARS-CoV-2 prevention model study using Peptide the peptide 346-001.

FIGS. 6A-B illustrate dose-response curves from an in vitro SARS-CoV-2 prevention model using Peptide the peptide 346-001.

FIGS. 7A-B illustrate dose-response curves from an in vitro SARS-CoV-2 prevention model using Peptide the peptide 346-002.

FIGS. 8A-B illustrate dose-response curves from an in vitro SARS-CoV-2 prevention model using Peptide the peptide 346-003.

FIGS. 9A-B illustrate dose-response curves from an in vitro SARS-CoV-2 prevention model using peptide 346-004.

FIGS. 10A-B illustrate dose-response curves from an in vitro SARS-CoV-2 prevention model using peptide 346-007.

FIGS. 11A-B illustrate dose-response curves from an in vitro SARS-CoV-2 prevention model using peptide 346-001.

FIGS. 12A-B illustrate dose-response curves from an in vitro SARS-CoV-2 prevention model using peptide 346-002.

FIGS. 13A-B illustrate dose-response curves from an in vitro SARS-CoV-2 prevention model using peptide 346-004.

FIGS. 14A-B illustrate dose-response curves from an in vitro SARS-CoV-2 prevention model using peptide 346-007.

FIG. 15 illustrates viral titer inhibition results from an in vitro SARS-CoV-2 prevention model using peptide 346-001.

FIG. 16 illustrates viral titer inhibition results from an in vitro SARS-CoV-2 prevention model using peptide 346-002.

FIG. 17 illustrates viral titer inhibition results from an in vitro SARS-CoV-2 prevention model using peptide 346-004.

FIG. 18 illustrates viral titer inhibition results from an in vitro SARS-CoV-2 prevention model using peptide 346-007.

FIGS. 19A-D illustrate in vitro HIV-1 inhibition of antiviral peptides derived from peptide 346-001 using an alanine scan.

FIGS. 20A-B illustrate the results from in vitro HIV-1 inhibition (mean+SD) studies using various antiviral peptides of the disclosure compared with peptide 346-001 at three concentrations; black fill, 0.1 μM peptide dosing concentration; checkered fill, 0.25 μM peptide dosing concentration; white fill, 1 μM peptide dosing concentration; ★, potency is lower than peptide 346-001; ★★, potency is equal to peptide 346-001; ★★★, potency is greater than peptide 346-001. The compounds are identified below the x-axis according to their peptide identifier (TABLE 1).

### DETAILED DESCRIPTION

The disclosure provides devices, systems and methods for treating, preventing, reducing the likelihood of having, reducing the severity of and/or slowing the progression of a condition in a subject. In various aspects, the disclosure provides materials and methods designed for the prevention and treatment of viral disease, specifically a viral disease caused by a virus, with the exclusion of respiratory viruses. Aspects of the materials and methods include, but are not limited to:
Peptides, or peptide prodrugs, or peptide conjugates, comprising 5-50 amino acids with antimicrobial properties;
Optionally, one or more other active pharmaceutical ingredients (APIs) (also referred to herein as “complementary agents”) used in combination with the above peptide; and/or
Drug delivery systems that result in a pharmacologically desirable amount of the peptide(s) (and, optionally API(s)) in the target anatomic compartment.

Thus, in various aspects, the disclosure provides methods of treating or preventing a viral disease (e.g., a non-respiratory viral disease). The methods comprise administering to a subject in need thereof a peptide comprising (or consisting essentially of, or consisting of) the sequence of peptide 346-001 set forth in TABLE 1 or variant thereof comprising one, two, or three amino acid substitutions. Alternatively, the methods comprise administering to a subject in need thereof variant of peptide 346-001 comprising four or five substitutions within the amino acid sequence of peptide 346-001. Additional peptide sequences for use in the context of the disclosure are presented in TABLE 1. The disclosure further provides compositions comprising any one or more of the peptides described herein. It will be appreciated that description of peptides herein is applicable to both descriptions of compositions and methods of treating or preventing a viral disease (e.g., a non-respiratory viral disease).

In various aspects, the peptide comprises the amino acid sequence of Formula 1:

(Formula 1; SEQ ID NO: 1)
X₁-X₂-X₃-X₄-X₅-X₆-X₇-X₈-X₉-X₁₀-X₁₁-X₁₂-X₁₃-X₁₄-X₁₅-X₁₆-X₁₇-
X₁₈,

wherein:
X₁ is S, K, D, A, N, M, T, or V; X₂ is W, I, Y, F, P, L, V, H, M, A, T, or S; X₃ is L, W, I, M, V, F, M, A, or T; X₄ is R, C, K, Q, H, P, or C; X₅ is D, R, I, E, N, or Y; X₆ is I, W, V, L, or F; X₇ is W, V, Y, F, P, L, V, H, or D; X₈ is D, S, E, N, Y, or S; X₉ is W, L, Y, F, P, L, V, or H; X₁₀ is I, V, L, or F; X₁₁ is C, S, A, P, M, H, or T; X₁₂ is E, D, S, Q, Y, or T; X₁₃ is V, F, I, L, A, F, or M; X₁₄ is L, V, I, M, or F; X₁₅ is S, D, A, N, T, Y, or P; X₁₆ is D, E, N, Y, or S; X₁₇ is F, W, Y, L, P, or H; and X₁₈ is K, E, R, H, R, P, or Q. The administration can be achieved by a range of parenteral routes of administration, including but not limited to: intravenous, subdermal, intramuscular, buccal (i.e., via the buccal mucosa), and topical, including but not limited applied to the skin, vaginally, and rectally. Optionally, the method comprises further administering one or more complementary agents (i.e., other APIs) suitable for, e.g., the treatment or prevention of a non-respiratory disease caused by viruses or related illness or symptom.

It will be appreciated that “treating” a disease or disorder does not require 100% abolition of the disease or disorder (i.e., complete reversal of the disease). Any degree of “treatment” is contemplated by the disclosure, including lessening of one or more symptoms of the disease, reduction in viral load, improvement in quality of life, and the like. Similarly, it will be appreciated that “preventing” a disease, in the context of the disclosure, does not require 100% inhibition of appearance of the disease. Any degree of reduction in the initial appearance of symptoms, inhibition of viral infection, prolonging relapse, inhibiting an initial surge of viral load, and the like, are contemplated. In various aspects, the viral non-respiratory disease is an HIV infection, e.g., an HIV-1 or HIV-2 infection. Alternatively, the viral non-respiratory disease may be an HSV infection, such as an HSV-1 or HSV-2 infection.

In the context of the disclosure, the subject is a mammal, which refers to any member of the class Mammalia, including, without limitation, humans and nonhuman primates such as chimpanzees and other apes and monkey species; farm animals such as cattle, sheep, pigs, goats and horses; domesticated mammals, such as dogs and cats; laboratory animals including rodents such as mice, rats and guinea pigs, and the like. The term does not denote a particular age or sex. Thus, adults (i.e., human subjects aged 18 years or more), children (i.e., human subjects aged one to eighteen years) and newborns (i.e., human subjects aged one year or less), whether male or female, are intended to be included within the scope of “subject.”

### Antiviral Peptides

The disclosure provides, e.g., peptides with antiviral properties and methods of use thereof. Optionally, the peptides have a length of five to fifty amino acid residues (e.g., five to eight amino acid residues; or eight to twelve amino acid residues; or twelve to twenty amino acid residues; or twenty to thirty five amino acid residues; or thirty five to fifty amino acid residues). In one exemplary aspect of the disclosure, the antimicrobial peptide comprises (or consists of, or consists essentially of) the amino acid sequence: SWLRDIWDWICEVLSDFK (SEQ ID NO: 3) peptide 346-001, TABLE 1). The amino acid sequence corresponds to a virucidal amphipathic α-helical peptide derived from the hepatitis C virus (HCV) NS5A membrane anchor domain, and is referenced herein peptide 346-001 (TABLE 1). The disclosure also contemplates use of a peptide derived from peptide 346-001. In this regard, the disclosure contemplates, e.g., a variant of Peptide 346-001 comprising, e.g., one, two, or three amino acid substitutions (such as conservative substitutions). In other aspects, the disclosure contemplates variants of peptide 346-001 comprising four or five amino acid substitutions. Optionally, the peptide comprises no more than seven, no more than six, no more than five, no more than four, no more than three, no more than two, or only one amino acid substitute compared to the sequence of peptide 346-001. For example, substitutions (e.g., conservative substitutions), deletions and additions may be made at non-critical residue positions within the selected peptide without substantially adversely affecting its biological activity. Modifications can be made at the receptor binding site(s) to adjust binding efficiency and specificity. In addition, changes may be made to select residues to increase peptide stability (e.g., replacing a cysteine residue with another amino acid, such as serine). The peptide can be optionally flanked and/or modified at one or both of the N- and C-termini, as desired.

Optionally, the peptide comprises the amino acid sequence of peptide 346-001 comprising one, two, or three substitutions (or more) selected from the following: the S at position 1 (X₁) is substituted with K, D, A, N, M, T, or V; the W at position 2 (X₂) is substituted with I, Y, F, P, L, V, H, M, A, T, or S; the L at position 3 (X₃) is substituted with W, I, M, V, F, M, A, or T; the R at position 4 (X₄) is substituted with C, K, Q, H, P, or C; the D at position 5 (X₅) is substituted with R, I, E, N, or Y; the I at position 6 (X₆) is substituted with W, V, L, or F; the W at position 7 (X₇) is substituted with V, Y, F, P, L, V, H, or D; the D at position 8 (X₈) is substituted with E, N, Y, or S; the W at position 9 (X₉) is substituted with L, Y, F, P, L, V, or H; the I at position 10 (X₁₀) is substituted with V, L, or F; the C at position 11 (X₁₁) is substituted with S, A, P, M, H, or T; the E at position 12 (X₁₂) is substituted with D, S, Q, Y, or T; the V at position 13 (X₁₃) is substituted with I, L, A, F, or M; the L at position 14 (X₁₄) is substituted with V, I, M, or F; the S at position 15 (X₁₅) is substituted with D, A, N, T, Y, or P; the D at position 16 (X₁₆) is substituted with E, N, Y, or S; the F at position 17 (X₁₇) is substituted with W, Y, L, P, or H; or the K at position 18 (X₁₈) is substituted with E, H, R, P, or Q. In various aspects, the peptide comprises the amino acid sequence of peptide 346-001 comprising a substitution at position 11 such that the cysteine is substituted with another amino acid (i.e., X₁₁ is not C). Optionally, the peptide comprises the amino acid sequence of peptide 346-001 wherein the C at position 11 (X₁₁) is substituted with S, A, P, M, H, or T. Also optionally, the peptide includes D forms of any of the amino acids described herein (e.g., all or a subset of the amino acids may be D-amino acids).

Optionally, the peptide comprises the amino acid sequence of peptide 346-001 comprising one, two, or three substitutions (or more, such as four or five substitutions), and the substitutions are selected from the following: the D at position 5 is substituted with I, E, or Y; the I at position 6 is substituted with W, V, L, or F; the W at position 7 is substituted with V, Y, F, L, H, or D; the D at position 8 is substituted with E, Y, or S; the I at position 10 is substituted with V, L, or F; the C at position 11 is substituted with S, A, M, H, or T; the V at position 13 is substituted with F, I, L, A, or M; the F at position 17 is substituted with W, Y, L, or H; or the K at position 18 is substituted with H, R, or P

In various aspects, the peptide has a sequence of Formula 1:

(Formula 1; SEQ ID NO: 1)
X₁-X₂-X₃-X₄-X₅-X₆-X₇-X₈-X₉-X₁₀-X₁₁-X₁₂-X₁₃-X₁₄-X₁₅-X₁₆-X₁₇-

wherein:
X₁ is S, K, D, A, N, M, T, or V; X₂ is W, I, Y, F, P, L, V, H, M, A, T, or S; X₃ is L, W, I, M, V, F, M, A, or T; X₄ is R, C, K, Q, H, P, or C; X₅ is D, R, I, E, N, or Y; X₆ is I, W, V, L, or F; X₇ is W, V, Y, F, P, L, V, H, or D; X₈ is D, E, N, Y, or S; X₉ is W, L, Y, F, P, L, V, or H; X₁₀ is I, V, L, or F; X₁₁ is C, S, A, P, M, H, or T; X₁₂ is E, D, S, Q, Y, or T; X₁₃ is V, I, L, A, F, or M; X₁₄ is L, V, I, M, or F; X₁₅ is S, D, A, N, T, Y, or P; X₁₆ is D, E, N, Y, or S; X₁₇ is F, W, Y, L, P, or H; and X₁₈ is K, E, H, R, P, or Q. Optionally, X₁ is S; X₂ is W; X₃ is L; X₉ is W; X₁₂ is E; X₁₄ is L; and X₁₅ is S.

The disclosure further provides a method of treating or preventing a viral non-respiratory disease, the method comprising administering to a subject in need thereof a peptide comprising the amino acid sequence S-W-L-X₄-X₅-X₆-X₇-X₈-W-X₁₀-X₁₁-E-X₁₃-L-S-D-X₁₇-X₁₈ (Formula 2; SEQ ID NO: 2), wherein X₄ is R, G, A, I, L, V, or F; X₅ is D, G, I, L, V, F, S, T, E, or Y; X₆ is I, A, W, V, L, or F; X₇ is W, A, V, Y, F, L, V, H, or D; X₈ is D, A, S, E, or Y; X₁₀ is I, A, V, L, or F; X₁₁ is C, S, A, W, M, H, W, R, V, or T; X₁₃ is V, F, I, L, A, or M; X₁₇ is F, W, Y, L, or H; and X₁₈ is K, R, H, A, P, or G. Optionally, the peptide comprises the amino acid sequence of peptide 346-001 having an amino acid substitution at four, three, two, or one amino acid position(s) selected from positions X₄, X₅, X₆, X₇, X₈, X₁₀, X₁₁, X₁₃, X₁₇, and X₁₈. For example, in some aspects at least one of the amino acid substitutions is at position X₄, X₅, or X₆. Alternatively or in addition, at least one of the amino acid substitutions is at position X₇, X₈, or X₁₀. Alternatively or in addition, at least one of the amino acid substitutions is at position X₁₃, X₁₇, or X₁₈. Optionally, the peptide comprises an amino acid substitution at position X₁₁. In various aspects, the peptide is not peptide 346-001.

With respect to the peptide of Formula 2, when X₄ is not R, it is G, A, I, L, V, or F. When X₅ is not D, it is G, I, L, V, F, S, T, E, or Y, optionally I, E, or Y. When X₆ is not I, it is A, W, V, L, or F, optionally W, V, L, or F. When X₇ is not W, it is A, V, Y, F, L, V, H, or D; optionally V, Y, F, L, V, H, or D, optionally A or F. When X₈ is not D, it is A, S, E, or Y, optionally, E, Y, or S. When X₁₀ is not I, it is A, V, L, or F, optionally V, L, or F. When X₁₁ is not C, it is S, A, W, M, H, W, R, V, or T, optionally S, A, M, H, or T. When X₁₃ is not V, it is F, I, L, A, or M. When X₁₇ is not F, it is W, Y, L, or H. When X₁₈ is not K, it is R, H, A, P, or G, optionally R, H, or P. In various aspects, the peptide comprises a sequence set forth in TABLE 1. The sequence may include L or D forms of the amino acids, or any combination thereof. The disclosure also contemplates peptides wherein the sequence set forth in TABLE 1 (e.g., Peptide 346-001) is reversed (i.e., a retropeptide, such as a peptide comprising the reversed sequence of Peptide 346-001 such that the N-terminus listed in TABLE 1 is the C terminus of the retropeptide).

Peptide sequences derived from peptide 346-001, and modifications thereof, may possess different, non-obvious pharmacologic properties relative to the parent sequence. These may consist of higher or lower in vitro and/or in vivo efficacy against one or more viruses that cause diseases. Other, exemplary properties that may be affected by modifying the peptide sequence include: in vitro stability; in vivo stability; in vivo half-life; protein binding; and cellular penetration. Non-limiting examples of peptide sequences against non-respiratory viruses are shown in TABLE 1.

In non-limiting aspects of the disclosure, the peptide contains overall features that impact its efficacy against viruses. One exemplary, non-limiting feature is associated with the peptide's α-helical structure. Without wishing to be bound by any particular theory, the α-helical structure can allow the peptide to interact with the viral membrane and disrupt its integrity, thereby releasing viral components such as capsids and exposing the viral genetic material to exonuclease degradation. The charge distribution along the peptide's α-helix, determined by the nature of the amino acid side-groups in the sequence, is an exemplary feature that may determine how the peptide associates, binds, and/or disrupts the viral particle. Another exemplary, non-limiting feature concerns the peptide's amphipathic nature, defined commonly in the art as possessing both hydrophilic and lipophilic properties. Without wishing to be bound by any particular theory, the peptide's amphipathicity can disrupt the ligand-host receptor interaction and viral escape from endosomes. The overall peptide hydrophobicity is another exemplary feature that can impact its antiviral properties by determining how it recognizes host-cellular components of virus membranes, possibly by their lipid composition. Another exemplary, non-limiting feature is associated with specific peptide-membrane protein interaction, such that retropeptides, peptides with scrambled hydrophobic or hydrophilic amino acids, or peptides comprised containing D-amino acids, but all based on the sequence of the parent, active peptide (e.g., peptide 346-001), display antiviral properties. In summary, there are a number of features that can, in certain embodiments, affect the antiviral peptide's interaction with the viral particle, specifically the viral membrane or another feature on the virus surface, causing disruption and leading to virus inactivation. In some cases, destabilization of physical linkages between the mature conical capsid core and the viral membrane, i.e., the core-membrane linkage can occur. In other cases, the shedding of the viral surface protein(s) responsible for entry into cells can be altered or shed.

Peptides are a series of amino acids connected one to the other by peptide bonds between the α-amino and α-carboxy groups of adjacent amino acids. Peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described. Although the peptide, in various aspects, is substantially free of other naturally occurring proteins and fragments thereof, in some embodiments the peptides can be synthetically conjugated to native fragments or particles.

In various aspects the peptides are optionally polymerized, each to itself, to form larger homopolymers, or with different peptides to form heteropolymers. In some instances, peptides will be combined in a composition as an admixture and will not be linked. The peptide can also be conjugated to lipid-containing molecules or to different peptides.

In various aspects of the disclosure, the peptide is chemically conjugated with a moiety that provides a functional or practical advantage. Non-limiting examples of such linked species, known in the art, include: polyethylene glycol, or other polymers to increase the in vivo residence time or half-life of the peptide; a small, biocompatible linker group that is amenable to high yielding bioconjugation reactions, so-called “click chemistry” linkers well known in the art (5-8) and fully incorporated herein; a biotin linker on the peptide that binds to streptavidin on another moiety, or other similar affinity-based linker systems known in the art, to facilitate in vivo detection or imaging of the peptide.

Linkages for homo- or hetero-polymers or for coupling to carriers can be provided in a variety of ways known in the art. For example, cysteine residues can be added at both the amino- and carboxy-termini, where the peptides are covalently bonded via controlled oxidation of the cysteine residues. Also useful are a large number of heterobifunctional agents which generate a disulfide link at one functional group end and a peptide link at the other, including N-succidimidyl-3-(2-pyridyl-dithio) proprionate (SPDP). This reagent creates a disulfide linkage between itself and a cysteine residue in one protein and an amide linkage through the amino on a lysine or other free amino group in the other.

In various aspects, the pharmacology of the peptide is enhanced by synthesizing a suitable prodrug; methods of prodrug synthesis are known in the art (9, 10). Prodrugs of the peptide can involve the C-terminal carboxy group, the tyrosine phenol group in tyrosyl peptides, and the N-terminal amino group. The peptide prodrug can have improved pharmacological properties when compared to the parent drug, such as improved bioavailability to the target compartment.

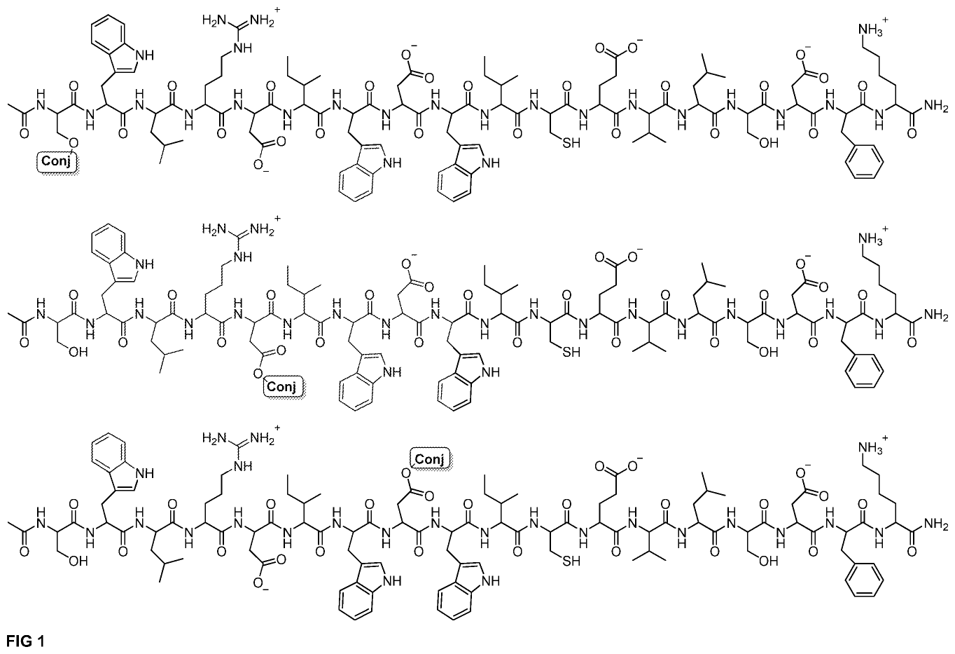

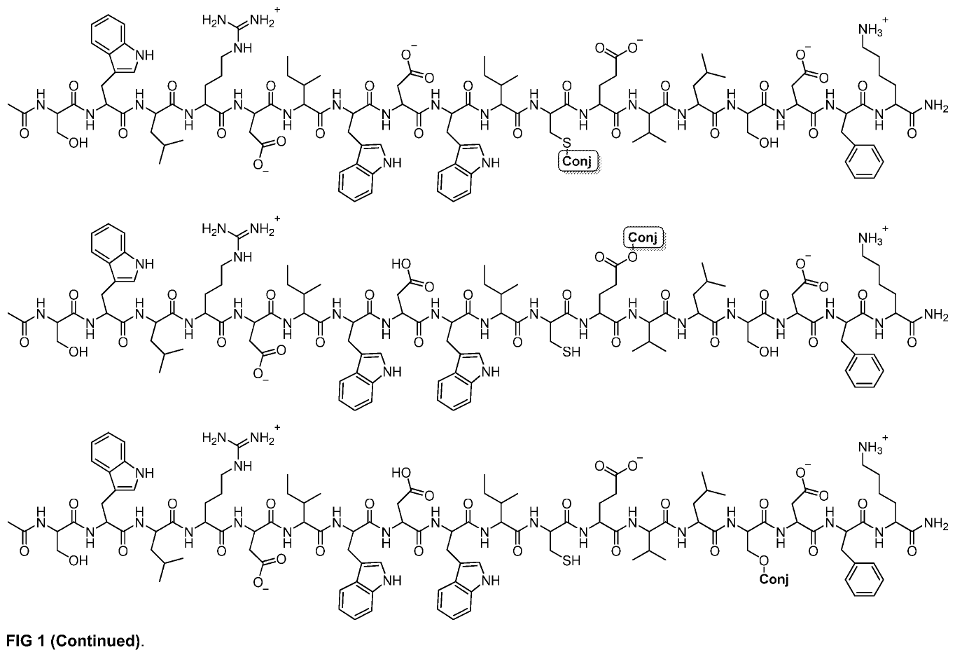

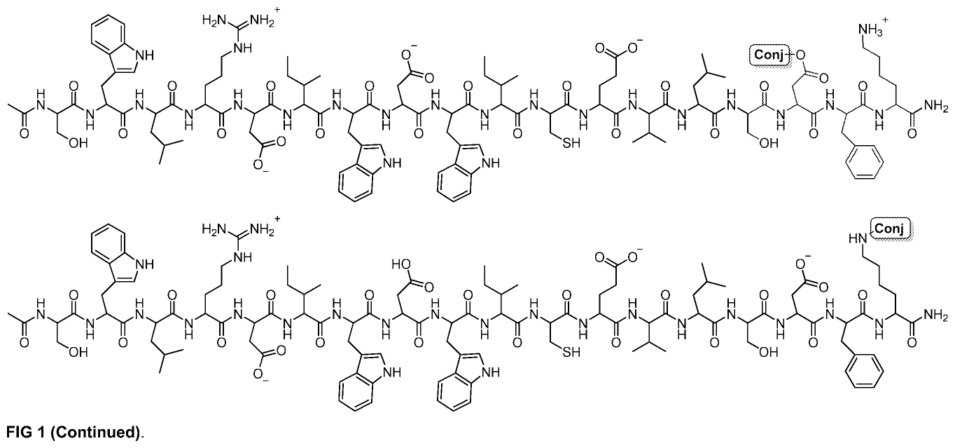

In various aspects of the disclosure, the pharmacology of the peptide is modified (e.g., enhanced) by synthesizing a suitable conjugate with a lipophilic moiety, or an acceptable salt thereof, as shown schematically in TABLE 2 using peptide 346-001 as an exemplary backbone. It is understood that this approach also applies to the other exemplary, non-limiting peptides shown in TABLE 1. In accordance with TABLE 2, peptide conjugation can occur in a variety of non-limiting strategies. Conjugation can be beneficial at the N-terminus, at the C-terminus, or at both termini. Conjugation can also be beneficial at the reactive side chains of the peptide, as shown in FIG. 1 using peptide 346-001 as an exemplary backbone. Any combination of these conjugation strategies can be used to enhance the properties of the parent peptide backbone. The conjugates are prepared using organic synthesis strategies, methods, and processes, many of which are known in the art.

The disclosure further contemplates peptides which are not conjugated with a lipophilic moiety.

In various aspects of the disclosure, the pharmacology of the peptide is modified (e.g., enhanced) by synthesizing a suitable conjugate with a second, complementary peptide. Conjugation can be beneficial at the N-terminus, at the C-terminus, or at both termini. Conjugation can also be beneficial at the reactive side chains of the peptide, as shown in FIG. 1 using peptide 346-001 as an exemplary backbone. Any combination of these conjugation strategies can be used to enhance the properties of the parent peptide backbone. The conjugates are prepared using organic synthesis strategies, methods, and processes, many of which are known in the art.

In various aspects, conjugation is achieved directly to the peptide. In various aspects, conjugation is achieved via one or more linker groups. Optionally, the linker, or spacer, consists of a single amino acid. Alternatively, the linker optionally comprises two or more amino acids, such as GSG, multiples thereof (GSG)ₙ, or GSGSGC (SEQ ID NO: 116), known in the art. As described herein, linkers can comprise peptides, polyether compounds (e.g., PEG), and/or combinations thereof. In applications where it is beneficial to have longer distance between the conjugate and the antiviral peptide, polyethylene glycol (PEG) chains of varying lengths may be employed.

In non-limiting examples, the complementary peptide moiety conjugated with the peptide described herein is a cell-targeting peptide, such as a cell-targeting peptide known in the art. Non-limiting examples of cell-targeting peptides include peptides derived from the V3 loop of gp120, the surface glycoprotein of HIV-1, such as the following nonlimiting exemplary sequences:

HIV-1 gp120 V3-A,
(SEQ ID NO: 117)
RKSIHIGPGRAFYTTG
HIV-1 gp120 V3-B,
(SEQ ID NO: 118)
RKGIRIGPGRAVYAAE

In another non-limiting example, the cell-targeting peptide comprises (or consists of) Tuftsin (TKPR (SEQ ID NO: 120)) or the canine analogs (TKPK (SEQ ID NO: 121), TKPKG (SEQ ID NO: 119)). In another non-limiting embodiment, the Tuftsin sequence is incorporated into the peptide 346 amino acid backbone. Non-limiting examples include:

(SEQ ID NO: 122)
X₁-X₂-X₃-X₄-X₅-X₆-X₇-X₈-X₉-X₁₀-X₁₁-X₁₂-X₁₃-X₁₄-T-K-P-R
(SEQ ID NO: 123)
X₁-X₂-X₃-X₄-X₅-X₆-X₇-X₈-X₉-X₁₀-T-K-P-R-X₁₅-X₁₆-X₁₇-X₁₈
(SEQ ID NO: 124)
T-K-P-R-X₅-X₆-X₇-X₈-X₉-X₁₀-X₁₁-X₁₂-X₁₃-X₁₄-X₁₅-X₁₆-X₁₇-X₁₈

Another non-limiting example, the complementary peptide moiety comprises a cell-penetrating peptide, such as a cell-penetrating peptide known in the art, such as the following nonlimiting exemplary sequences:

Tat48-60,
(SEQ ID NO: 125)
GRKKRRQRRRPPQ
HIV-1 Tat-(49-57),
(SEQ ID NO: 126)
RKKRRQRRR
HIV-1 Rev-(34-50),
(SEQ ID NO: 127)
TRQARRNRRRRWRERQR
FHV Coat-(35-49),
(SEQ ID NO: 128)
RRRRNRTRRNRRRVR
Penetratin,
(SEQ ID NO: 129)
RQIKIWFQNRRMKWKK
DPV3,
(SEQ ID NO: 130)
RKKRRRESRKKRRRES
CADY,
(SEQ ID NO: 131)
GLWRALWRLLRSLWRLLWRA
MAP,
(SEQ ID NO: 132)
KLALKLALKALKAALKA
Maurocalcine,
(SEQ ID NO: 133)
GDCLPHLKLCKENKDCCSKKCKRRGTNIEKRCR
pVEC,
(SEQ ID NO: 134)
LLIILRRRIRKQAHAHSK
TP10,
(SEQ ID NO: 135)
AGYLLGKINLKALAALAKKIL
TP2,
(SEQ ID NO: 136)
PLIYLRLLRGQF
Pep-1,
(SEQ ID NO: 137)
KETWWETWWTEWSQPKKKRKV
Pep-7,
(SEQ ID NO: 138)
SDLWEMMMVSLACQY
p28,
(SEQ ID NO: 139)
LSTAADMQGVVTDGMASGLDKDYLKPDD
Transportan,
(SEQ ID NO: 140)
GWTLNSAGYLLGKINLKALAALAKKIL
C105Y,
(SEQ ID NO: 141)
CSIPPEVKFNKPFVYLI
KFGF,
(SEQ ID NO: 142)
AAVLLPVLLAAP
Polyarginine,
(R)ₙ 4 ≤ n ≤ 12

In non-limiting examples, the sequence of the complementary peptide moiety is reversed (i.e., a retropeptide, such that the N-terminus of the complementary peptides listed above is the C-terminus of the retropeptide). In other, nonlimiting examples, one or more amino acids in the complementary peptide sequence are D-amino acids.

In one various aspects, conjugation is achieved directly to the peptide. In various aspects, conjugation is achieved via one or more linker groups. Optionally, the linker, or spacer, consists of a single amino acid. Alternatively, the linker optionally comprises two or more amino acids, such as GSG, multiples thereof (GSG)ₙ, or GSGSGC (SEQ ID NO: 116), known in the art. As described herein, linkers can comprise peptides, polyether compounds (i.e., PEG), and/or combinations thereof. In applications where it is beneficial to have longer distance between the conjugate and the antiviral peptide, polyethylene glycol (PEG) chains of varying lengths. PEG is a typically biologically inert chemical that confers greater water solubility to peptides with which it is incorporated as constituent chemical group. PEG is non-toxic and non-immunogenic, hydrophilic, and highly flexible. In additional embodiments, the linker comprises one or more polyethylene glycol oligomer moieties having a formula of —(OCH₂CH₂)ₘ—, wherein m is an integer 1 to 24. In one exemplary embodiment, m is 24-1,000. In another embodiment, m is 1,000-5,000.

In accordance with this aspect of the present disclosure, linkers may be an amino acid, such as, but not limited to, lysine, serine, aspartic acid, glutamic acid, or cysteine.

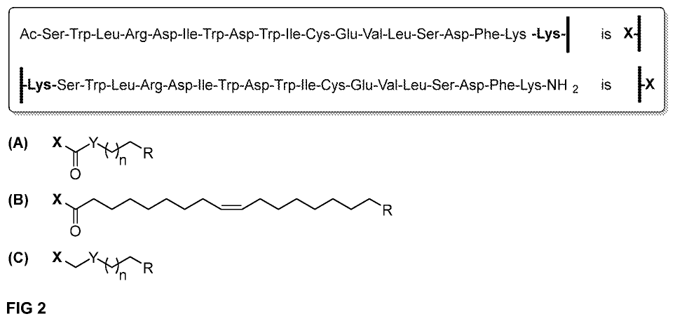

When lysine is used as a linker, either as a single amino acid or as part of a larger linker comprising two or more amino acids, synthetic spacers (e.g., PEG) or a combination thereof, the amino side-chain can be used for conjugation as shown in FIG. 2 using the peptide 346-001 backbone as a non-limiting example. Suitable conjugates can be carbonyl compounds, as depicted in FIGS. 2 (A) and (B). In such non-limiting embodiments, Y may be O, S, NR₁ (where R₁ is H, alkyl, or aryl), P(O)R₂ [where R₂ is alkyl, alkyl-aryl (e.g., benzyl), aryl, OH, O-alkyl, O-alkyl-aryl, O-aryl, NR₁-alkyl, NR₁-alkyl-aryl, or NR₁-aryl], or CR₂R₃ [where R₂ and or R₃ are alkyl, alkyl-aryl (e.g., benzyl), aryl, O-alkyl, O-alkyl-aryl, O-aryl, or NR₁-alkyl, NR₁-alkyl-aryl, or NR₁-aryl]. In other words, as described herein, exemplary, non-limiting conjugates as shown in FIG. 2 can be classified as amides, carbamates, ureas, thiocarbamates, phosphoramidates, and phosphonates. The alkyl chain length, n, is 1-50 and R is alkyl, aryl, or derivatives thereof, such as PEG. In one non-limiting example, palmitic acid is conjugated with the lysine linker; i.e., Y=CH₂, n=13, R=CH₃. In non-limiting embodiments, the conjugation moiety is unsaturated, such as oleic acid (R=CH₃) as shown in FIG. 2 (B), or linoleic acid, and comprises one or more double bonds. In other non-limiting examples, the lysine NH₂ side chain is conjugated via a methylene group, as shown in FIG. 2 (C), and Y can be O, S, NR₁, P(O)R₂, or CR₂R₃.

In non-limiting examples the moiety conjugated to X via the O═C—Y— group as described above and in FIG. 2 (A), or via the CH₂—Y— group as described above in FIG. 2 (C) comprises one or more cholesterol groups, or derivatives thereof. In further non-limiting examples, the moiety comprises of one or more tocopherol groups, or derivatives thereof. Tocopherols constitute a series of related benzopyranols (or methyl tocols) that occur in plant tissues and vegetable oils and are powerful lipid-soluble antioxidants. These compounds are produced by plants and other oxygenic photosynthetic organisms, such as algae and some cyanobacteria, and are essential components of the diet of animals, and collectively they are termed “vitamin E”. The antiviral peptide conjugate of the present disclosure includes tocopherol, a tocopherol derivative, or pharmaceutically acceptable salt thereof. Examples of tocopherol that may be used in the present aspect include, but are not limited to, α-tocopherol, β-tocopherol, γ-tocopherol, and δ-tocopherol. Examples of useful tocopherol derivatives or pharmaceutically acceptable salts thereof include, but are not limited to, tocotrienols, tocopheroxyl radical, 8α-alkyldioxytocopherone, tocopherol quinone, tocopherol hydroquinone, D,L-tocopherol, D,1-tocopheryl acetate, α-tocopheryl acetate, α-tocopheryl nicotinate, and α-tocopheryl succinate. In further non-limiting examples, the moiety comprises one or more dihydrosphingosine groups, one or more sphingosine groups, or derivatives thereof. In further non-limiting examples, the moiety comprises one or more phospholipid groups, or derivatives thereof.

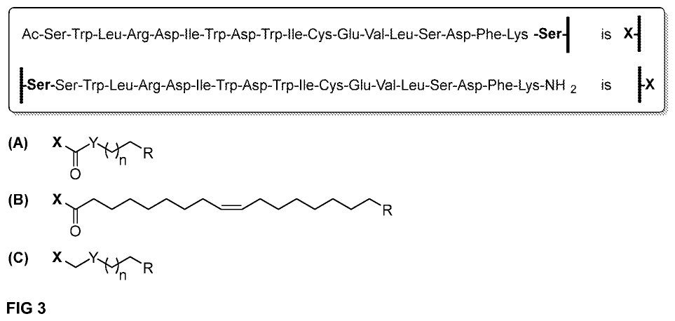

When serine is used as a linker, either as a single amino acid or as part of a larger linker comprising two or more amino acids, synthetic spacers (e.g., PEG) or a combination thereof, the hydroxy side-chain can be used for conjugation as shown in FIG. 3 using the peptide 346-001 backbone as a non-limiting example. Suitable conjugates consist of carbonyl compounds, as depicted in FIGS. 3 (A) and (B). In such non-limiting embodiments, Y may be O, S, NR₁ [where R, is H, alkyl, alkyl-aryl (e.g., benzyl), or aryl], P(O)R₂ [where R₂ is alkyl, alkyl-aryl (e.g., benzyl), aryl, OH, O-alkyl, O-alkyl-aryl, O-aryl, NR₁-alkyl, NR₁-alkyl-aryl, or NR₁-aryl], or CR₂R₃ [where R₂ and or R₃ are H, alkyl, alkyl-aryl (e.g., benzyl), aryl, O-alkyl, O-alkyl-aryl, O-aryl, or NR₁-alkyl, NR₁-alkyl-aryl, or NR₁-aryl]. In other words, as described herein, exemplary, non-limiting conjugates as shown in FIG. 2 can be classified as esters, carbonates, carbamates, thiocarbonates, phosphoramidates, and phosphonates. The alkyl chain length, n, is 1-50 and R is alkyl, aryl, or derivatives thereof, such as PEG. In one non-limiting example, palmitic acid is conjugated with the serine linker; i.e., Y=CH₂, n=13, R=CH₃. In non-limiting embodiments, the conjugation moiety is unsaturated, such as oleic acid (R=CH₃) as shown in FIG. 3 (B), or linoleic acid, and comprises or more double bonds. In other non-limiting examples, the serine OH side chain is conjugated via a methylene group, as shown in FIG. 3 (C), and Y can be O, S, NR₁, P(O)R₂, or CR₂R₃.

In non-limiting examples the moiety conjugated to X via the O═C—Y— group as described above and in FIG. 3 (A), or via the CH₂—Y— group as described above in FIG. 3 (C) comprises one or more cholesterol groups, or derivatives thereof. In further non-limiting examples, the moiety comprises one or more tocopherol groups, or derivatives thereof. In further non-limiting examples, the moiety comprises one or more dihydrosphingosine groups, one or more sphingosine groups, or derivatives thereof. In further non-limiting examples, the moiety comprises one or more phospholipid groups, or derivatives thereof.

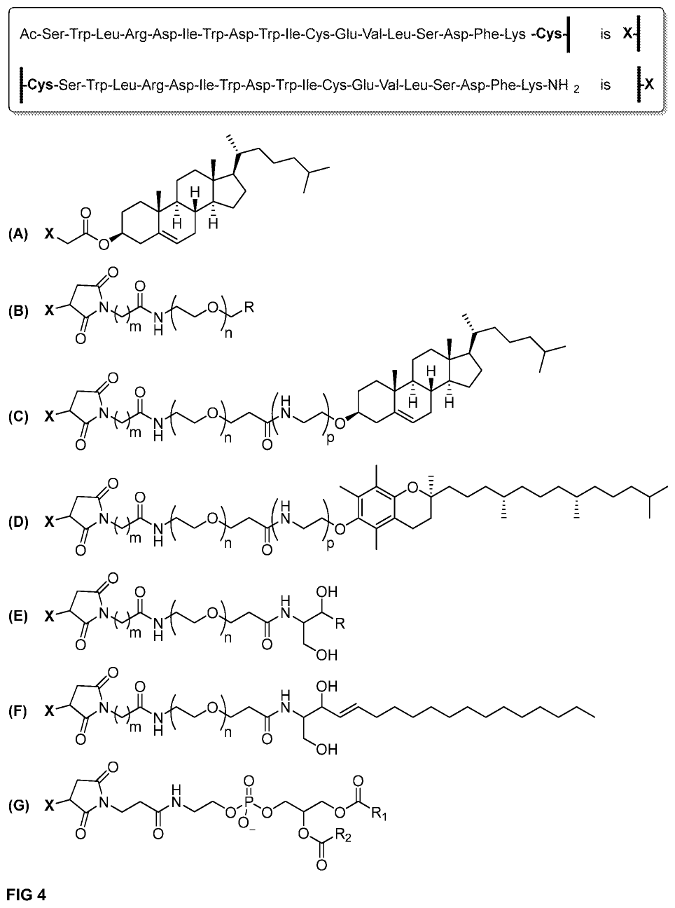

In general, thiol groups present in cysteine (or cysteine derivative) side-chains or terminal groups can be reacted with reagents possessing thiol-reactive functional groups using reaction schemes known in the art. Exemplary thiol-reactive functional groups include, without limitation, iodoacetamides, maleimides, and alkyl halides. Non-limiting examples of such conjugation schemes using a terminal cysteine linker are shown in FIG. 4. Cysteine can be used as a linker, either as a single amino acid or as part of a larger linker comprising two or more amino acids, synthetic spacers (e.g., PEG) or a combination thereof. FIG. 4 uses the peptide 346-001 backbone as a non-limiting example. Exemplary, non-limiting embodiments shown in FIG. 4 include: (A) derivatives of cholesterol, or pharmaceutically acceptable salts there-of; (B) maleimide derivatives of PEG, or pharmaceutically acceptable salts there-of, where m is 1-12, n is 1-12, and R is H, alkyl, or aryl; (C) maleimide derivatives of cholesterol, or pharmaceutically acceptable salts there-of, where m is 1-12, n is 1-12, and p is 1-12; (D) maleimide derivatives of tocopherol, or pharmaceutically acceptable salts there-of, where m is 1-12, n is 1-12, and p is 1-12; (E) maleimide derivatives of dihydrosphingosine, or pharmaceutically acceptable salts there-of, where m is 1-12, n is 1-12, and R is H, alkyl, or aryl; (F) maleimide derivatives of sphingosine, or pharmaceutically acceptable salts there-of, where m is 1-12, and n is 1-12; (G) maleimide derivatives of phospholipids, or pharmaceutically acceptable salts thereof, where R is H, alkyl, or aryl.

The peptides of the disclosure can be prepared in a wide variety of ways. In various aspects, the peptide is desirably small while maintaining substantially all of the virucidal activity of the large peptide. The peptides can be prepared synthetically or by recombinant DNA technology using, e.g., methods known in the art. The peptides can be synthesized in solution or on a solid support in accordance with conventional techniques. Various automatic synthesizers are commercially available and can be used in accordance with known protocols.

Compositions are also provided that comprise a peptide (or multiple peptides) of the disclosure. For example, in various aspects, the disclosure provides a composition comprising a peptide comprising the sequence: S-W-L-X₄-X₅-X₆-X₇-X₈-W-X₁₀-X₁₁-E-X₁₃-L-S-D-X₁₇-X₁₈ (Formula 2; SEQ ID NO: 2), wherein X₄ is R, G, A, I, L, V, or F; X₅ is D, G, I, L, V, F, S, T, E, or Y; X₆ is I, A, W, V, L, or F; X₇ is W, A, V, Y, F, L, V, H, or D; X₈ is D, A, S, E, or Y; X₁₀ is I, A, V, L, or F; X₁₁ is C, S, A, W, M, H, W, R, V, or T; X₁₃ is V, F, I, L, A, or M; X₁₇ is F, W, Y, L, or H; and X₁₈ is K, R, H, A, P, or G, wherein the peptide is not peptide 346-001. Optionally, the peptide comprises the amino acid sequence of Peptide 346-001 having an amino acid substitution at four, three, two, or one amino acid position(s) selected from positions X₄, X₅, X₆, X₇, X₈, X₁₀, X₁₁, X₁₃, X₁₇, and X₁₈. In various aspects, at least one of the amino acid substitutions is at (a) position X₄, X₅, or X₆; (b) position X₇, X₈, or X₁₀; or (c) position X₁₃, X₁₇, or X₁₈. Optionally, the peptide comprises an amino acid substitution at position X₁₁. Examples of substitutions include, but are not limited to: if X₄ is not R, it is G, A, I, L, V, or F; if X₅ is not D, it is G, I, L, V, F, S, T, E, or Y; optionally I, E, or Y; if X₆ is not I, it is A, W, V, L, or F; optionally W, V, L, or F; if X₇ is not W, it is A, V, Y, F, L, V, H, or D; optionally V, Y, F, L, V, H, or D; if X₈ is not D, it is A, S, E, or Y; optionally, E, Y, or S; if X₁₀ is not I, it is A, V, L, or F; optionally V, L, or F; if X₁₁ is not C, it is S, A, W, M, H, W, R, V, or T; optionally S, A, M, H, or T; if X₁₃ is not V, it is F, I, L, A, or M; if X₁₇ is not F, it is W, Y, L, or H; and/or if X₁₈ is not K, it is R, H, A, P, or G; optionally R, H, or P. Also optionally, one or more amino acids are D-amino acids. In various aspects, the peptide is formulated with an additional peptide, a liposome, an adjuvant and/or a pharmaceutically acceptable carrier. Liposomes can also be used to increase the half-life of the peptide composition. Liposomes useful in the context of the present disclosure include emulsions, foams, micelles, insoluble monolayers, liquid crystals, phospholipid dispersions, lamellar layers and the like. In these preparations the peptide to be delivered is incorporated as part of a liposome, alone or in conjunction with a molecule that binds to a suitable receptor, or with other therapeutic or immunogenic compositions. Thus, liposomes filled with a desired peptide of the disclosure can be directed to the site of cells infected with the virus, where the liposomes then deliver the selected therapeutic/immunogenic peptide compositions. Liposomes for use in the context of the disclosure may be formed from standard vesicle-forming lipids, which generally include neutral and negatively charged phospholipids and a sterol, such as cholesterol. The selection of lipids is generally guided by consideration of, e.g., liposome size and stability of the liposomes in vivo. A variety of methods are available for preparing liposome, many of which are described in the art.

In another aspect, the peptide is chemically or physically attached to polymeric micro- or nanoparticles, a number of which are known in the art. Nonlimiting examples include biodegradable, biocompatible microspheres and nanospheres, where nonlimiting examples of resorbable synthetic polymers include poly(lactic acids), poly(glycolic acids), poly(lactic-co-glycolic acids), poly(caprolactones) (PCLs), and mixtures thereof. In some embodiments, the peptide is dispersed inside the polymer particles using emulsion techniques well known in the art. In other embodiments, the N-terminus of the peptide is conjugated to the surface of the polymer particles via free carboxy groups. Alternatively, the peptide C-terminus is coupled to free carboxy groups on the surface of the polymer particles via a suitable linker group using synthetic chemistry approaches well-known in the art. The purposes of the polymer micro- or nanoparticles include, but are not limited to: monodispersity for efficient delivery to target anatomic regions, and mucoadhesive properties to affect retention times of the agent.

### Complementary Agents

In addition to the peptide disclosed above, one or more complementary agents are optionally administered to the subject. “Complementary” is intended to mean that the agents are supportive in achieving the desired pharmacological or biological effect (e.g., alleviating one or more symptoms of a viral non-respiratory disease, alleviating one or more side-effects of the disease, addressing a separate illness or disorder, or addressing an associated disruption in normal functioning of the subject).

In various aspects, the complementary agent(s) possesses antimicrobial properties. Nonlimiting examples of complementary antimicrobial agents include:
Antiretroviral agents, including, but not limited to nucleoside reverse transcriptase inhibitors (NRTIs, e.g., tenofovir disoproxil fumarate, tenofovir alafenamide, emtricitabine, lamivudine), nucleoside reverse transcriptase translocation inhibitors (NRTTI, e.g., islatravir), non-NRTIs (e.g., rilpivirine, etravirine, doravirine), integrase inhibitors (INSTIs, e.g., raltegravir, dolutegravir, cabotegravir, elvitegravir), protease inhibitors (PIs, e.g., tipranavir, darunavir, atazanavir), and capsid inhibitors (CIs, e.g., lenacapavir),
Antiviral agents, including, but not limited to valaciclovir, acyclovir, famciclovir, ganciclovir, and remdesivir,
Broad-spectrum antimicrobial agents, including, but not limited to L-lactic acid and D-lactic acid, as well as probiotic bacteria, such as lactobacilli,
Antibacterial agents, including, but not limited to metronidazole, clindamycin, and tinidazole,
Antifungal agents, including, but not limited to fluconazole, voriconazole, and amphotericin B, and
Antiparasitic agents, including, but not limited to ivermectin.

In various aspects, the complementary microbicide component is a small molecule therapeutic. For example, the complementary agent may be a CD4-mimetic compound that binds HIV vaginally, as described by Madani et al. (11) incorporated herein in its entirety. In another aspect of the disclosure, the complementary microbicidal compound is a biologic therapeutic (e.g., a protein therapeutic or a nucleic acid-based therapeutic).

In another aspect, the complementary antiviral agent comprises a protein. A non-limiting example of an antiviral protein is an antiviral protein belonging to the lectin family, such as griffithsin (GRFT), cyanovirin-N(CV-N) and scytovirin (SVN), described in (12), which is incorporated by reference herein in its entirety. In another nonlimiting example, the antiviral agent is a neutralizing antibody (bNAb), such as a broadly neutralizing antibody, a non-limiting example of which is VRC01 that possesses activity against various HIV-1 isolates (13). Other, next generation bNAbs that are more potent against HIV or neutralize other viruses such as HSV or HPV, or bacteria, such as multidrug-resistant Neisseria gonorrhoeae are also included herein as part of the disclosure.

In some cases, the complementary pharmaceutically active substance is an antibacterial agent. In some cases, the antibacterial agent is a biologic with activity against multidrug-resistant Neisseria gonorrhoeae, such as those described in the art (e.g., 14-17, incorporated herein by reference).

In some cases, the complementary agent is an agent that affects immune and fibrotic processes. Non-limiting examples of agents that affect immune and fibrotic processes include, but are not limited to, inhibitors of Rho-associated coiled-coil kinase 2 (ROCK2), for example, KD025 (Kadmon) and didemnins (e.g., plitidepsin).

In various aspects, the complementary agent is a contraceptive. As used herein, the term “contraceptive” refers to an active agent that prevents conception or pregnancy. In various aspects, the contraceptive is a hormonal contraceptive or a non-hormonal contraceptive. In various aspects, the complementary agent is a hormonal contraceptive. Non-limiting examples of hormonal contraceptives include estrogens or progestins, e.g., estradiol, etonogestrel, levonorgestrel, medroxyprogesterone acetate, segesterone acetate, norethindrone, and progesterone. In various aspects, the complementary agent is a non-hormonal contraceptive. In various aspects, the non-hormonal contraceptive comprises multivalent antibodies (e.g., IgGs) with high agglutination potencies for trapping vigorously motile sperm, or a small molecule, such as an inhibitor of soluble adenylyl cyclase (sAC:ADCY10), essential for male fertility.

The complementary agents described herein can be administered alone (as part of a therapeutic regimen including administration of the peptide) or in combination with other complementary agents (and administration of the peptide). In some cases, the formulations described herein comprise more than one pharmaceutically active substance. In some cases, the formulations described herein comprise a combination of pharmaceutically active substances.

In certain aspects of the disclosure, a complementary agent is chemically linked with the peptide to generate peptide-drug conjugates. This strategy is an effective conjugation strategy for targeted delivery and improved pharmacological outcomes as described, for example, in the review by Wang et al. (18), incorporated herein by reference in its entirety and in particularly in regard to its discussion of conjugates.

### Multipurpose Prevention Technologies

The disclosure contemplates use of the peptide described herein in the context of multipurpose prevention technologies, including delivery of the peptide using the delivery devices and methods described below. The oral, subdermal, intramuscular, rectal, and vaginal delivery of antiviral (e.g., antiretroviral (ARV)) drugs has shown clinical promise in protecting women from HIV infection. However, the effectiveness of this pre-exposure prophylaxis (PrEP) strategy has been severely limited by adherence to the dosing regimen. Multipurpose prevention technologies (MPTs) (19) are an integrated biomedical approach that provides dual (or more) protection (20-22), usually from HIV infection, along with other sexually transmitted infections (STIs), and unintended pregnancy. The MPT strategy exploits synergies in contraceptive demand for STI protection (23), significantly improving user compliance relative to single-purpose antiviral products (24).

The inclusion of a contraceptive feature in MPTs can motivate product uptake and use for HIV PrEP (22, 23, 25-27), thereby improving effectiveness. Women can receive dual protection discreetly, even if their stated intention is to address just one health need, because of pressures from their sociocultural context (e.g., HIV stigma) or relationships. The inclusion of a contraceptive feature in MPT intravaginal rings (IVRs) can strongly motivate product uptake and use for HIV PrEP, thereby improving effectiveness. When asked about preference for single indication products versus MPTs, the overwhelming majority of reproductive age women in various settings, geographical locations, and demographic groups expressed a preference for an MPT. Providing access to MPTs therefore may indirectly improve both contraception and HIV/STI prevention coverage (28, 29).

All MPTs disclosed in the art share a number of common features. At least one of the agents delivered by the MPT via IVR acts in the cervicovaginal tissues or systemic circulation. For example, the two most prominent IVR MPTs under development involve the delivery of levonorgestrel (LNG) as the contraceptive agent and either dapivirine (DPV) (30) or tenofovir (TFV) (31) as the antiviral agent against HIV. Both products are undergoing clinical evaluation in sub-Saharan Africa (32-34). In these MPT IVRs the contraceptive agent acts systemically and possibly in the cervicovaginal tissues, while the antiviral drugs (DPV and TFV) are believed to act in the cervicovaginal tissues. None of the drugs are active in the cervicovaginal fluids.

A number of other vaginal MPT products are under development and aim to prevent unwanted pregnancy using hormonal contraceptives such as a progestin only (e.g., LNG) or a progestin-estrogen combination, such as etonogestrel and ethinyl estradiol. Hormonal contraception is known in the art to result in real and perceived side effects, including altered bleeding patterns, weight gain, mood swings and depression, headaches and nausea (35). Many women would prefer a non-hormonal method that does not require use immediately before or after sex. In the context of an MPT that protects against sexual HIV acquisition and unintended pregnancy, there is growing concern, largely based on injectable depot medroxyprogesterone acetate (DMPA) (36, 37), that a LNG-only regimen as part of an MPT may lead to increased risk of HIV-1 acquisition (38-42) The use of a vaginally active non-hormonal contraceptive is desirable as it avoids these potential risks associated with exogenous hormones. The disclosure contemplates use of the peptide described herein in the context of vaginal MPT products.

When drugs are delivered from a vaginal product, they partition into the cervicovaginal fluids and, from there, into the tissues and finally systemic circulation. There are considerable drug concentration gradients across these anatomic compartments, with the cervicovaginal fluid having drug concentrations order(s) of magnitude higher than the other compartments, depending on the agent's physicochemical properties. It therefore is theoretically beneficial to use an agent against HIV or other STIs that is active in the cervicovaginal fluids.

The disclosure contemplates use of a vaginal MPT product, such as a product disclosed herein, to deliver the peptide described herein, optionally with one or more other agents, all of which are active in the cervicovaginal fluids. The agents are optionally microbicidal against HIV and other microorganisms leading to STIs and provide non-hormonal contraception.

An MPT drug formulation can include essentially any therapeutic, prophylactic, or diagnostic agent that would be useful for delivery to an anatomic compartment. In the disclosure, the MPT drug product must contain at least one or more antiviral peptides disclosed herein and one or more contraceptive agents. One or more additional complementary agents, as described above, can be delivered in combination with the antiviral peptide, as determined by the application.

In one preferred aspect, the non-hormonal contraceptive is active against sperm. For example, ferrous gluconate causes spermiostasis (43, 44). In another, non-limiting example, the non-hormonal contraceptive is a small molecule, such as an inhibitor of soluble adenylyl cyclase (sAC:ADCY10), essential for male fertility (45). Other non-limiting examples of male, non-hormonal contraceptives known in the art target EPPIN, a surface protein on human spermatozoa that has an essential function in reproduction (46, 47), and cyclin-dependent kinase 2 (CDK2) (48), included herein in their entirety. In another embodiment, the non-hormonal contraceptive consists of multivalent IgGs with high agglutination potencies for trapping vigorously motile sperm (49, 50).

### Administration Methods

The active agent(s) described above (i.e., the antiviral peptide and complementary agents, when present) are administered to the mammalian subject using any suitable route of delivery. Pharmaceutical compositions of the present disclosure can be administered orally, by inhalation, via a pulmonary route of administration, intranasally (including intranasal instillation), topically, transdermally, intrapleurally, intraperitoneally, by application to a mucous membrane, parenterally, topically, intravenously, subcutaneously, intraperitoneally, or by intramuscular administration.

Delivery of the antiviral peptide(s) and complementary agents, if applicable, is optionally achieved via a pulsatile means as known in the art; e.g., oral tablet, intravenous injection, subcutaneous injection, intramuscular injection, intravitreal injection, topical cream, vaginal tablet, vaginal film, vaginal insert, vaginal gel, vaginal cream, rectal suppository, rectal enema, or rectal gel.

Alternatively, delivery of the antiviral peptide(s) and complementary agents may be achieved using a sustained release or long-acting system or drug delivery device as known in the art; e.g., injectable nanoparticles (subcutaneous, intramuscular, intravitreal), injectable microparticles (subcutaneous, intramuscular, intravitreal), microneedles and microneedle arrays, subdermal implants, intramuscular implants, intravaginal rings. Non-limiting examples of devices suitable for delivery of the antiviral peptide(s) and complementary agents include those disclosed in PCT/US21/60815, WO 2021/108722, and WO 2021/071974, the disclosure of each of which is incorporated herein by reference.

For example, a suitable drug delivery device can comprise a scaffold comprising one or more biocompatible materials, one or more chambers containing a plurality of cells, one or more membranes, and one or more nutrient supplementation systems. In the context of the disclosure, the cells may produce the peptide described herein. In some cases, the drug delivery device is adapted for intravaginal use. In some cases, the one or more biocompatible materials comprise one or more thermoplastic polymers, one or more elastomers, one or more biocompatible metals, or combinations thereof. Non-limiting examples of suitable biocompatible materials include silicone, polyurethane, poly(ethylene-co-vinyl acetate) (EVA), or a combination thereof. Non-limiting examples of suitable cells include bacterial cells, fungal cells, mammalian cells, or a combination thereof. The cells may produce the peptide described herein, or may produce a different active agent suitable for use with the peptide described herein. Non-limiting examples of suitable materials for the membrane or membranes include polyester, polypropylene, polycarbonate, polyethylene terephthalate (PET), anisotropic materials, polysulfone (PSF), microfiber or nanofiber mats, polyimide, tetrafluoroethylene/polytetrafluoroethylene (PTFE), expanded polytetrafluoroethylene (ePTFE), poly(ethylene-co-vinyl acetate) (EVA), polyacrylonitrile, polyethersulfone, acrylic resin, cellulose acetate, cellulose nitrate, polyamide, hydroxylpropyl methyl cellulose (HPMC), or a combination thereof. Non-limiting examples of suitable nutrient supplementation systems comprise nutrients, growth factors, hormones, vitamins, O₂-generating agents, pH buffering agents, cell culture media, antibiotics, or a combination thereof.

For example, a suitable drug delivery device can comprise (a) one or more kernels comprising one or more active pharmaceutical ingredients (APIs), such as the peptide described herein; and (b) one or more skins comprising a continuous membrane; wherein the one or more kernels and/or the skin comprises defined pores, and wherein the pores are not produced mechanically. In some embodiments, the reservoir kernel comprises a paste comprising one or more APIs. In some embodiments, the kernel comprises a fiber-based carrier and/or a porous sponge. In some embodiments, the device further comprises a shape adapted to be disposed within the body of a patient. For example, the device can be capsule-shaped, or can be in the shape of a torus. In some embodiments, the device comprises one or more cylindrical core elements disposed within a first skin, wherein the core elements comprise a kernel and optionally a second skin. In some embodiments, the skin comprises a rate-limiting skin. Non-limiting examples of suitable materials for the skin include poly(dimethyl siloxane), silicone, one or more polymers, and/or metal, where the polymer can be, without limitation, poly(ether), poly(acrylate), poly(methacrylate), poly(vinyl pyrolidone), poly(vinyl acetate), poly(urethane), cellulose, cellulose acetate, poly(siloxane), poly(ethylene), poly(tetrafluoroethylene) (such as expanded poly(tetrafluoroethylene) or ePTFE) and other fluorinated polymers, poly(siloxanes), copolymers thereof, or combinations thereof, poly(amides), poly(esters), poly(ester amides), poly(anhydrides), poly(orthoesters), polyphosphazenes, pseudo poly(amino acids), poly(glycerol-sebacate), poly(lactic acids), poly(glycolic acids), poly(lactic-co-glycolic acids), poly(caprolactones) (PCLs), PCL derivatives, amino alcohol-based poly(ester amides) (PEA), poly(octane-diol citrate) (POC), copolymers thereof, or mixtures thereof.

For example, a suitable drug delivery device can comprise a buccal implant device configured to provide sustained drug delivery, the buccal implant device comprising: a body having a non-cylindrical geometry, the body adapted to be disposed within the oral mucosa of a patient; and one or more active pharmaceutical ingredients (e.g., peptide described herein) disposed within the body. In some embodiments, the buccal implant device comprises: a body adapted to be disposed within the oral mucosa of a patient; means for guiding the body into or out of the oral cavity of a patient; and one or more active pharmaceutical ingredients disposed within the body. In some embodiments, the device comprises a matrix, reservoir, or hybrid design comprising one or more thermoplastic polymers, elastomer materials, or metals suitable for pharmaceutical use and a therapeutically effective amount of one or more active pharmaceutical ingredients. For example, the device can be a buccal implant device configured to provide sustained drug delivery, the buccal implant device comprising: a body adapted to be disposed within the oral mucosa of a patient; means for guiding the body into or out of the oral cavity of a patient; and one or more active pharmaceutical ingredients disposed within the body. Non-limiting examples of suitable shapes for the device include a saw shape, a saber shape, and a ribbed shape. In some embodiments, the body is tapered, e.g., having a single taper, two tapers, or three tapers. In some embodiments, the body has one or more curved edges configured to direct the implant into a specific orientation within the oral cavity. In some embodiments, the one or more curved edges form a point at an end of the body. In some embodiments, the device further comprises a hole formed in the body and/or a loop coupled to the hole to guide the body into or out of the oral cavity. In some embodiments, the device comprises expanded polytetrafluoroethylene (ePTFE) having microscopic pores. In some embodiments, the device comprises a rate-limiting skin comprising, without limitation, expanded polytetrafluoroethylene (ePTFE).

### Excipients and Manufacturing Considerations

The disclosure contemplates a composition comprising one or more of the peptides described herein and one or more pharmaceutically acceptable excipients. Pharmaceutically acceptable excipients are known in the art and may include, e.g., viscosity modifiers, bulking agents, surface active agents, dispersants, disintegrants, osmotic agents, diluents, binders, anti-adherents, lubricants, glidants, pH modifiers, antioxidants and preservants, and other non-active ingredients of the formulation intended to facilitate handling and/or affect the release kinetics of the drug.

In some embodiments, the binders and/or disintegrants may include, but are in no way limited to, starches, gelatins, carboxymethylcellulose, croscarmellose sodium, methyl cellulose, ethyl cellulose, hydroxymethyl cellulose, hydroxyethyl cellulose, hydroxypropyl cellulose, hydroxypropylmethyl cellulose, hydroxypropylethyl cellulose, hydroxypropylmethyl cellulose, microcrystalline cellulose, polyvinylpyrrolidone, polyethylene glycol, sodium starch glycolate, lactose, sucrose, glucose, glycogen, propylene glycol, glycerol, sorbitol, polysorbates, and/or colloidal silicon dioxide. In certain embodiments, the anti-adherents or lubricants may include, but are in no way limited to, magnesium stearate, stearic acid, sodium stearyl fumarate, and/or sodium behenate. In some embodiments, the glidants may include, but are in no way limited to, fumed silica, talc, and/or magnesium carbonate. In some embodiments, the pH modifiers may include, but are in no way limited to, citric acid, lactic acid, and/or gluconic acid. In some embodiments, the antioxidants and preservants may include, but are in no way limited to ascorbic acid, butylated hydroxytoluene (BHT), butylated hydroxyanisole (BHA), cysteine, methionine, vitamin A, vitamin E, sodium benzoate, and/or parabens.

In some embodiments, excipients can stabilize biomolecules with respect to degradation or loss of biological activity using approaches known to those skilled in the art (51). Certain excipients stabilize biomolecules by creating a “water-like” environment in the dry state through hydrogen bonding interactions, e.g., sugars (52) and amino acids (53). Other excipients create a glassy matrix that provides hydrogen bonding and immobilized the biomolecules to prevent aggregation that leads to loss of biologic activity (e.g., trehalose, inulin). Still other excipients can stabilize the pH in the implant formulation (e.g., buffer salts). Finally, surfactants can reduce the concentration of the biomolecules at the air-water interface during drying processes of formulation, decreasing shear stress and insoluble aggregate formation, and allowing the previously described stabilization mechanisms to occur throughout the drying process.

Solid formulations for dry powder inhalers (DPIs) can be prepared by a variety of methods, many of which are well known in the art. These include, but are not limited to spray drying, spray-freeze drying, supercritical fluid technology, solvent precipitation method, double emulsion/solvent evaporation technique, particle replication in nonwetting templates, and lyophilization. The resulting polydisperse mixtures can be refined further by specialized milling techniques. Jet-milling of drugs and excipients under nitrogen gas with a nano-jet milling machine is one non-limiting exemplary method known in the art for creating nanoparticles meant for pulmonary drug delivery.

### Methods

The disclosure provides materials and methods for delivery of an antiviral peptide (and, optionally, one or more complementary agents) to a subject for the purposes of treating, preventing, reducing the likelihood of having, reducing the severity of and/or slowing the progression of a medical condition in a subject. In a preferred aspect, the medical condition is a viral infection, or exposure to a virus. In this regard, the disclosure provides a method of treating or preventing a viral non-respiratory disease. By “viral non-respiratory disease” is meant a disease whose primary etiology is not a viral infection of the respiratory system of a patient, e.g., an infection of the upper respiratory system and/or the lower respiratory system. In particular, the acquisition of the virus is not predominantly through the respiratory system, e.g., through the nasal passages, larynx, trachea, lungs, and/or bronchi.

In various aspects, the peptide is delivered to a subject using an implant system which delivers the peptide (and one or more other APIs) to a body compartment, thereby treating, preventing, reducing the likelihood of having, reducing the severity of and/or slowing the progression of a non-respiratory viral disease in a subject. In some cases, the anatomic compartment is the vagina. In other cases, the target body compartment is systemic circulation. A long-acting drug delivery system reduces problems associated with patient adherence to regimens that involve frequent dosing from, for example, the subcutaneous, intramuscular, or buccal compartments.

In some cases, a subject in need of treatment for a disease or disorder disclosed herein, such as an infectious disease, is symptomatic for the disease or disorder. In some cases, a subject in need of treatment for a disease or disorder disclosed herein, such as an infectious disease, is asymptomatic for the disease or disorder. A subject in need of treatment for a disease or disorder disclosed herein can be identified by a skilled practitioner, such as without limitation, a medical doctor or a nurse. In some cases, the antiviral peptide is used to prevent a viral disease in the subject.

Illustrative, non-restrictive examples of non-respiratory viral diseases contemplated by the disclosure are provided below in summary form. Based on these examples, one skilled in the art can adapt the disclosed technology to other applications. One skilled in the art would recognize whether such applications involve topical drug delivery (e.g., certain vaginal implant devices such as IVRs) or systemic drug delivery (e.g., subdermal or intramuscular implant devices).
HIV prevention, wherein the method optionally comprises administration of a complementary agent which is a suitable antiretroviral agent (e.g., a biologic), or a vaccine and/or adjuvant;
HIV treatment, wherein the method optionally comprising administering using a complementary agent which is a suitable antiretroviral agents (e.g., a biologic);
Sexually transmitted infections (STIs), including but not limited to prevention or treatment, both active and chronic active, wherein the method optionally comprises administering a complementary agent which is a suitable antimicrobial agent. Illustrative, but not limiting examples of STIs include: gonorrhea, chlamydia, lymphogranuloma venereum, syphilis, including multidrug-resistant (MDR) organisms, hepatitis C virus, and herpes simplex virus;
Hepatitis B virus (HBV) prevention or treatment, both active and chronic active;
Herpes simplex virus (HSV) and varicella-zoster virus (shingles) Zoster/Shingles, prevention or treatment, both active and chronic active;
Cytomegalovirus (CMV) and congenital CMV infection, prevention or treatment, both active and chronic active.
   In some embodiments, the prevention of a viral infection is complemented by the prevention of unintended pregnancy (i.e., contraception), optionally using an MPT approach as described above. In an MPT approach, the treatment or prevention of a viral infection can be complemented by the treatment or prevention of a non-viral infection, for example, bacterial vaginosis (BV), as well as other microbial dysbiotic vaginal states, including both active and chronic active.

### EXAMPLES

### Example 1—Formulation 1 (Intravaginal Administration—On Demand)

For on-demand intravaginal administration, antiviral peptide from the 346 library (TABLE 1) is formulated as a vaginal film product. The films are produced by the solvent casting method, known in the art, and consist of poly(vinyl alcohol) (PVA, 67 kDa). To a solution of PVA (25% w/w) in deionized water is added slowly with magnetic stirring the antiviral peptide 0.01-100 mM, preferably 0.5-10 mM and preservatives known in the art to stabilize peptides (e.g., histidine and polysorbate 20). The pH of the solution is adjusted to an optimal pH for peptide stability, typically in the 6.0-7.5 range. The solution is stirred magnetically to ensure dissolution of all components and homogenous distribution, while removing entrapped air bubbles. The solution then is cast onto a plastic substrate (e.g., polyester) on a glass plate using a die press. The film is allowed to dry at room temperature and then is cut into sections of predetermined dimensions using a scalpel.

In another example, a non-hormonal contraceptive from the sAC inhibitor class is included in the film formulation at 0.1-1,000 μM, preferably 10-100 μM, in combination with the antiviral peptide to form a multipurpose prevention technology.

### Example 2—Formulation 2 (Intravaginal Administration—Long-Acting)

For long-acting intravaginal administration, antiviral peptide from the 346 library (TABLE 1) is formulated as an intravaginal ring product, using designs known in the art to be suitable for the delivery of such agents. The device is engineered to deliver the antiviral peptide at daily release rates in the 0.01-10 mg d⁻¹ range, preferably in the 0.2-2 mg d⁻¹. Preferably, the antiviral peptide is delivered in combination with a non-hormonal contraceptive from the sAC inhibitor class controlled independently to deliver the contraceptive at daily release rates in the 0.01-10 mg d⁻¹ range, preferably in the 0.1-1 mg d⁻¹. The combination forms an example of a form a multipurpose prevention technology.

### Example 3—Formulation 3 (Intrarectal Administration—On Demand)

For on-demand intrarectal administration, antiviral peptide from the 346 library (TABLE 1) is formulated as rectal enema product. The peptide is dissolved in normal saline, or 50% v/v normal saline in deionized water. The concentration of the peptide is 0.01-100 mM, preferably 0.1-5 mM. Sodium hydroxide (5 M) or hydrochloric acid (5 M) is added to bring the solution near neutral pH. Alternately, the peptide powder is added to a sodium bicarbonate solution (1 mg mL⁻¹) and sodium chloride powder is added at a final concentration of 0.9% w/v to produce a saline-based enema. The final osmolarity of the formulation is 50-500 mOsm kg⁻¹, preferably 100-300 mOsm kg⁻¹.

In another example, the antiretroviral drug tenofovir (TFV), or a suitable prodrug of tenofovir, also is dissolved in the rectal enema product along with the peptide as described above to create a combination product. The concentration of TFV is 0.5-50 mg mL⁻¹, preferably 0.5-5 mg mL⁻¹. Prodrugs of TFV are formulated at the same molar equivalent concentration.

In another example, the above ingredients are premixed and stored in solid form to produce the enema formulation on demand by the addition of water. This can hold the advantage of a longer shelf-life in smaller product package volume and mass.

### Example 4—Substitution Analysis

An alanine scan was performed on peptide 346-001 to identify amino acids in the sequence that are important for virucidal efficacy. An established HIV-1 in vitro model was used in the first round of screening.

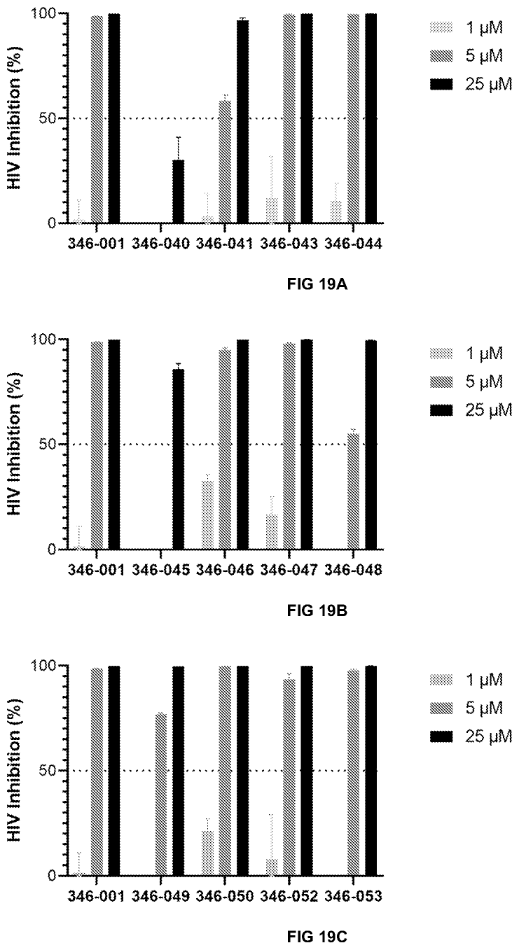

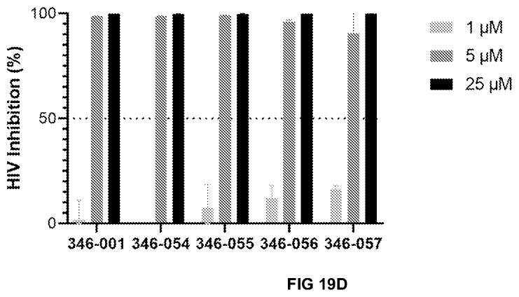

HeLa TZM reporter cells (expressing CD4, CCR4, CCR5, and β-galactosidase) were grown to 100,000 cells in a 96-well format, in triplicate. An HIV-1 (primary R5 JR-CSF) stock was grown to in human peripheral blood mononuclear cells (PBMCs) and standardized by HIV-1 p24 ELISA. The peptide solution (10 μL, diluted from a DMSO stock) and HIV-1 aliquot (10 μL) were mixed and incubated at 37° C. for 0.5 h. Cells were treated with the peptide-virus mixture (20 μL), or control, in triplicate at each dose by mixing with media (150 μL). The culture was incubated for 6 h at 37° C. and then washed. The challenge endpoint was at 48 h, and viral infection was analyzed in supernatant by β-galactosidase quantification. Negative controls consist of the most concentrated DMSO solution used above (no drug). Positive control consists of peptide 346-001, prepared as above. The results are shown in FIG. 19A-19D and show that peptide 346-001 tolerates substitutions within the amino acid sequence and retains virucidal activity.

### Example 5—In Vitro SARS-CoV-2 Prevention Model (Study 1)

Dosing concentrations of peptide 346-001 and peptide 346-009 (negative control) were prepared from DMSO stock solutions by dilution with 1× minimum essential medium (MEM) at predetermined dilution levels. The resulting dosing solutions (100 μL) were added in triplicate to freshly aspirated wells containing 80% confluent VERO E6 cultures, prepared in 48-well format (18 wells were used for each compound). Three wells of the 18 were treated with the highest concentration, but were not infected with virus to serve as toxicity controls. The remaining wells (12) including four used to test toxicity of the DMSO vehicle (0.5% stock in MEM medium, 100 μL/well) without infection; four wells similarly treated and then infected to determine impact of the DMSO on the virus; and 4 wells that were not treated (provided 100 μL of medium) and infected to show maximal viral infection for the study.

The treated plate was transferred to the BLS3 facility where the designated wells were challenged with 10⁴ TCID₅₀ SARS-CoV-2 (2019-nCoV/USA-WA1/2020 strain) in 100 μL of serum-free MEM. The time between drug and virus addition to the wells was ca. 20 min. The plate was incubated for 48 h at 37° C. Each well was then lysed by addition of 200 μL of MagNAPure™ External Lysis solution without aspiration. The 400 μL of lysed material was subjected to automated MagNAPure96 IVD DNA and viral NA extraction method. Quantification of viral titers was completed using RT-qPCR assays optimized for clinical diagnostics. For each sample, copies of the SARS-CoV-2 envelope “E” gene, open reading frame 8 “orf8” gene and the human RNAseP “RP” gene were quantified on CFX Real-Time Systems™, or equivalent. Primer sequences and amplimer specifics are provided below. The two viral targets indicated impact on viral infection while the RP copy number was a surrogate for cellular toxicity.

E Amplimer: 113 bp (forward and reverse primers in bold)

(SEQ ID NO: 143)
ACAGGTACGTTAATAGTTAATAGCGT-
ACTTCTTTTTCTTGCTTTCGTGGTATTCTTGCTAGTTACACTAGCCATCCTTACTGCGCTT
CGATTG-TGTGCGTACTGCTGCAATAT

Base pairs: 26277 to 26389; Sequence ID: LC528233.1

orf 8 Amplimer: 91 bp (forward and reverse primers in bold)

(SEQ ID NOL 144)
AATCAGCACCTTTAATTGAATTG-
TGCGTGGATGAGGCTGGTTCTAAATCACCCATTCAGTACATCGA-
TATCGGTAATTATACAGTTTCCTG

Base pairs: 28059 to 28149; Sequence ID: LC528233.1

RNAse P Amplimer: 64 bp (forward and reverse primers in bold)

(SEQ ID NO: 145)
AGA TTT GGA CCT GCG AGC G
(SEQ ID NO: 1460
GAG CGG CTG TCT CCA CAA GT

Base pairs: 28 to 92; GenBank Sequence ID: NM_006413.5

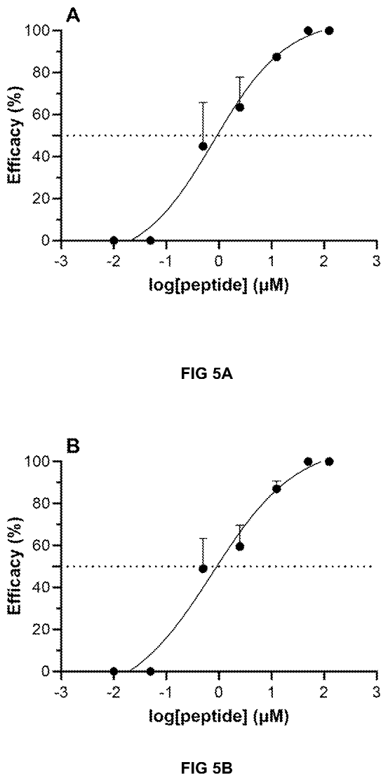

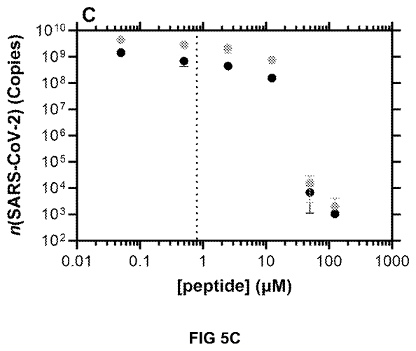

Results from this study are shown in FIGS. 5A-5C. Under these experimental conditions, the antiviral peptide displayed a surprising, high potency against SARS-CoV-2 infection. At 50 μM dosing concentration, a reduction on viral copy numbers of >5 orders of magnitude was observed, and >7 orders of magnitude at 125 μM dosing concentration (FIG. 5C).

### Example 6—In Vitro SARS-CoV-2 Prevention Model (Study 2)

Eleven dosing concentrations of peptides 346-001, 346-002, 346-003, 346-004, 346-005, 346-007, 346-008, and 346-009 (negative control) were prepared as freshly created DMSO stock solutions by dilution with 1×MEM at predetermined dilution levels. The resulting dosing solutions (50 μL) were added in quadruplicate to freshly aspirated wells containing 60% confluent VERO E6 cultures, prepared in 96-well format (44 wells were used for each compound). A final 4 wells were not treated (50 μL MEM) serving as maximal viral infection controls.

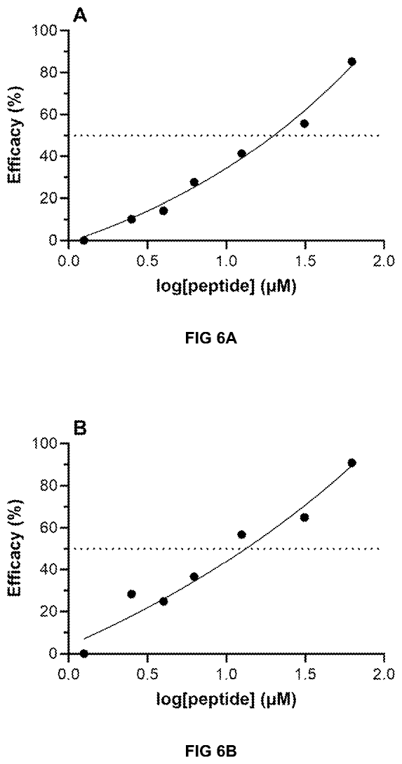

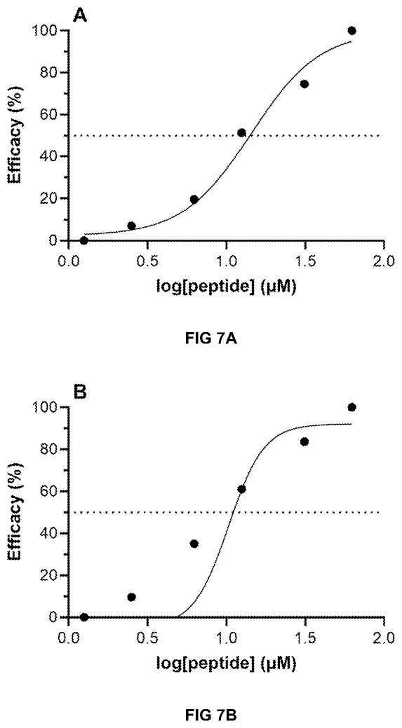

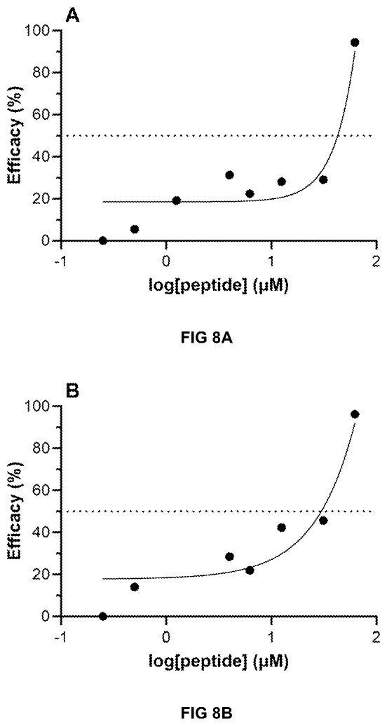

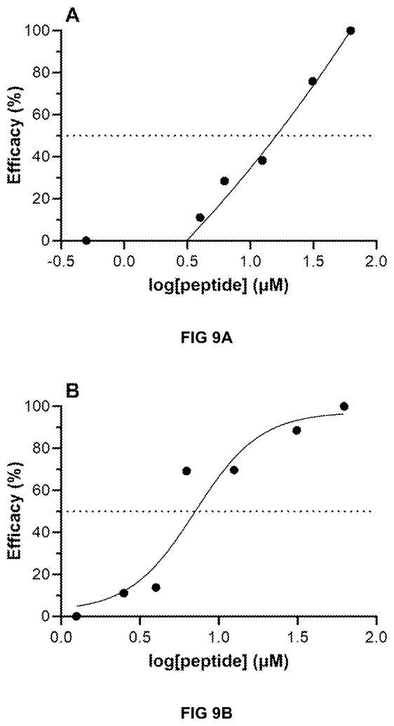

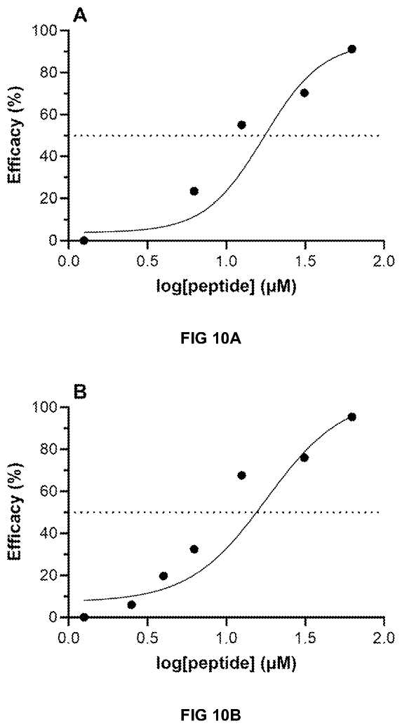

The treated plates were transferred to the BLS3 facility where the designated wells were challenged with 10³ TCID₅₀ SARS-CoV-2 (2019-nCoV/USA-WA1/2020 strain) in 50 μL of serum-free MEM. The time between drug and virus addition to the wells was ca. 40 min. The plates were incubated for 48 h at 37° C. Each well was then lysed by addition of 100 μL of MagNAPure™ External Lysis solution without aspiration. The 200 μL of lysed material was subjected to automated MagNAPure96™ IVD DNA and viral NA extraction method. Quantification of viral titers was completed as described in EXAMPLE 3. Results from this study are shown in FIG. 6A-10B. Comparison of different antiviral peptides based on peptide 346-001 afforded surprising results in terms of the dose-response relationships against SARS-CoV-2. The potencies of the peptides tested varied depending on the sequence, but all provided robust activity against SARS-CoV-2. The shapes of the dose-response curves varied substantially, even between peptides of identical sequences, but using L-(peptide 346-001) versus D-(peptide 346-002) amino acids, as shown in FIGS. 6A/6B and 7A/7B, respectively. Peptide 346-003 displayed an unexpected and unusual dose response curve shape (FIGS. 8A and 8B).

### Example 7—In Vitro SARS-CoV-2 Prevention Model (Study 3)

Eleven dosing concentrations of peptides 346-001, 346-002, 346-004, and 346-007 were prepared as freshly created DMSO stock solutions by dilution with 1×MEM at predetermined dilution levels. The resulting dosing solutions (50 μL) were added in quadruplicate to freshly aspirated wells containing 80% confluent VERO E6 cultures, prepared in 96-well format (44 wells were used for each compound). A final 4 wells were not treated (50 μL MEM) serving as maximal viral infection controls.

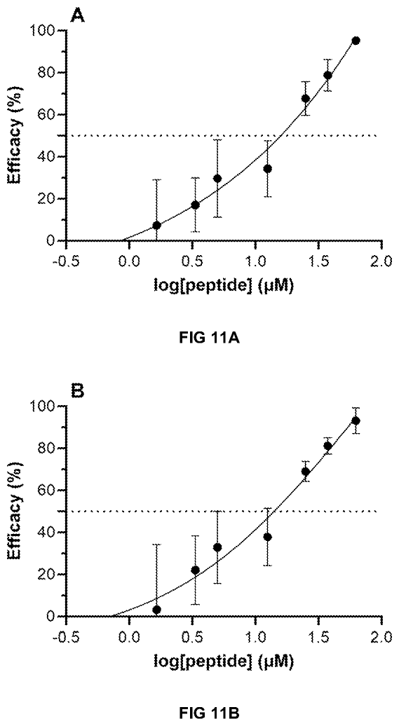

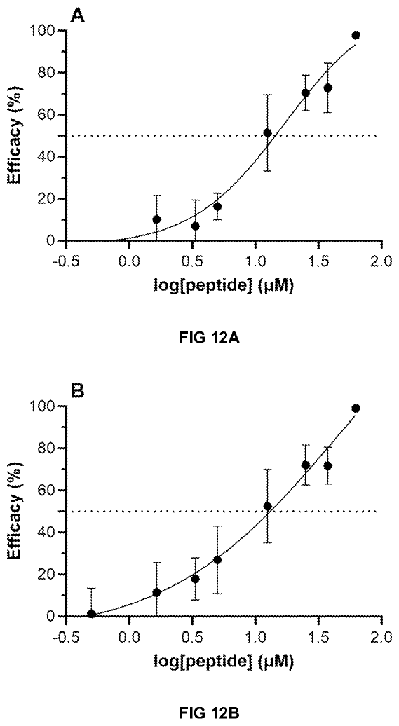

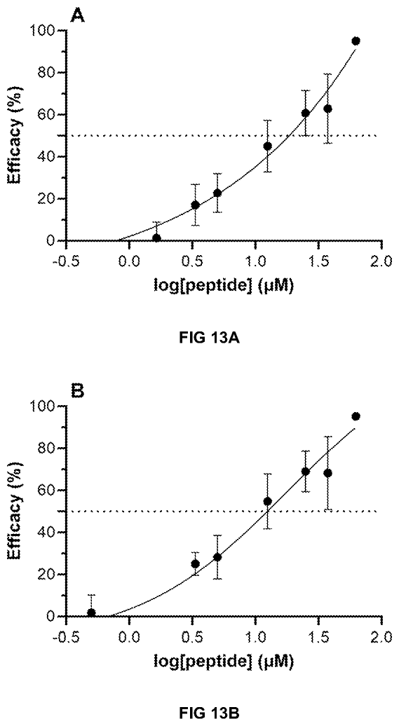

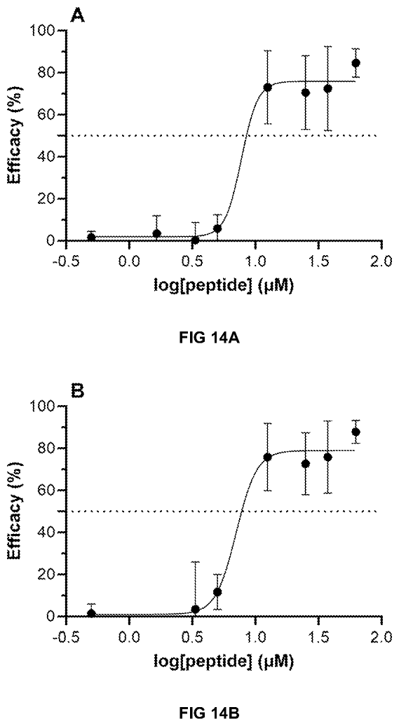

The treated plates were transferred to the BLS3 facility where the designated wells were challenged with 10³ TCID₅₀ SARS-CoV-2 (2019-nCoV/USA-WA1/2020 strain) in 50 μL of serum-free MEM for VERO E6 wells. The time between drug and virus addition to the wells was ca. 40 min. The plates were incubated for 48 h at 37° C. Each well was then lysed by addition of 100 μL of MagNAPure™ External Lysis solution without aspiration. The 200 μL of lysed material was subjected to automated MagNAPure96™ IVD DNA and viral NA extraction method. Quantification of viral titers was completed as described for EXAMPLE 3. Results from this study are shown in FIG. 11A-14B. Comparison of different antiviral peptides based on peptide 346-001 afforded surprising results in terms of the dose-response relationships against SARS-CoV-2. All of the peptides tested provided robust activity against SARS-CoV-2. All peptides tested except peptide 346-007 displayed similar dose-response relationships and antiviral potencies. However, peptide 346-007 led to a steep dose response relationship with ca. double the potency of the other peptides tested in this example (FIGS. 14A and 14B).

### Example 8—In Vitro SARS-CoV-2 Prevention Model (Study 4)

Eleven dosing concentrations of peptides 346-001, 346-002, 346-004, and 346-007 were prepared as freshly created DMSO stock solutions by dilution with 1×MEM at predetermined dilution levels. The resulting dosing solutions (5 μL) were added in quadruplicate to air-liquid interfaced human nasal epithelial cells (NEC) Transwell cultures, prepared in 96-well format (44 wells were used for each compound).

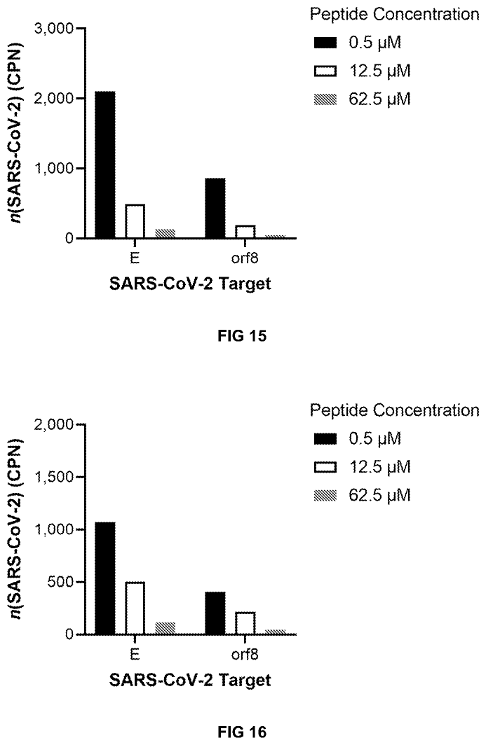

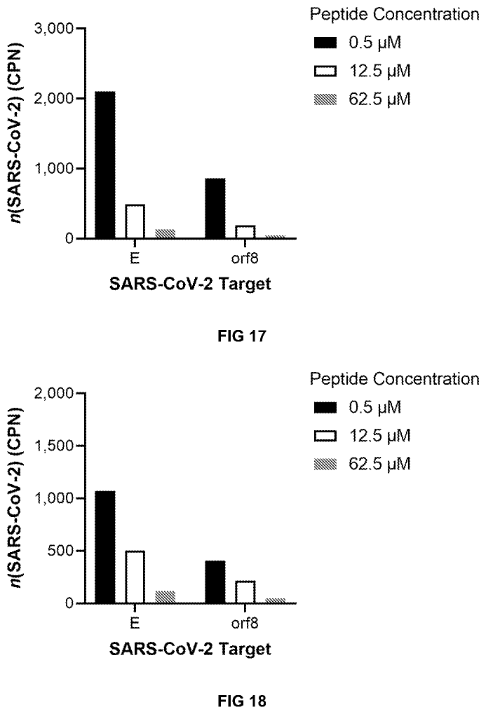

The treated plates were transferred to the BLS3 facility where the designated wells were challenged with 10³ TCID₅₀ SARS-CoV-2 (2019-nCoV/USA-WA1/2020 strain) in 5 μL of serum-free MEM. The time between drug and virus addition to the wells was ca. 40 min. The plates were incubated for 48 h at 37° C. Each well was then lysed by addition of 200 μL of MagNAPure™ External Lysis solution in the apical chamber. The 200 μL of lysed material was subjected to automated MagNAPure96™ IVD DNA and viral NA extraction method. Quantification of viral titers was completed as described for Study 1. Results from this study are shown in FIG. 15-18. An innovative human nasal epithelial culture system with an apical air interface was used in lieu of VERO cells (i.e., non-human primate origin) in this example to provide a more real-world evaluation of select antiviral peptides. Surprisingly, the potency of all peptides tested was under the 12.5 μM dosing concentration.

### Example 9—In Vitro HIV-1 Prevention Model

Sixty-seven (67) compounds from a library of peptides included in TABLE 1 were evaluated for inhibition of HIV-1 IIIB in TZM-bl-FcRI cells. The compounds were tested at three (3) concentrations with six (6) replicates each for efficacy and toxicity for 48 hours in this model. Virus was diluted 1:128, based on a previous titration. The test article was dissolved in DMSO to generate a stock solution, and dosing solutions were prepared by dilution of the stock solution using the appropriate volume of PBS. These dosing solutions were mixed with predetermined volumes of the virus suspension, based on the titration data, and were incubated for 30 min at 37° C./5% CO₂.

Following incubation, the virus/compound mixture was added to pre-plated TZM-bl-FcRI cells and the necessary volume of assay media (based on the titration). The culture was incubated for six hours under the above conditions and then washed to remove residual virus and compound. The cultures were incubated for an additional 48 hours at 37° C./5% CO₂ before measuring β-galactosidase in the wells using a chemiluminescent endpoint.

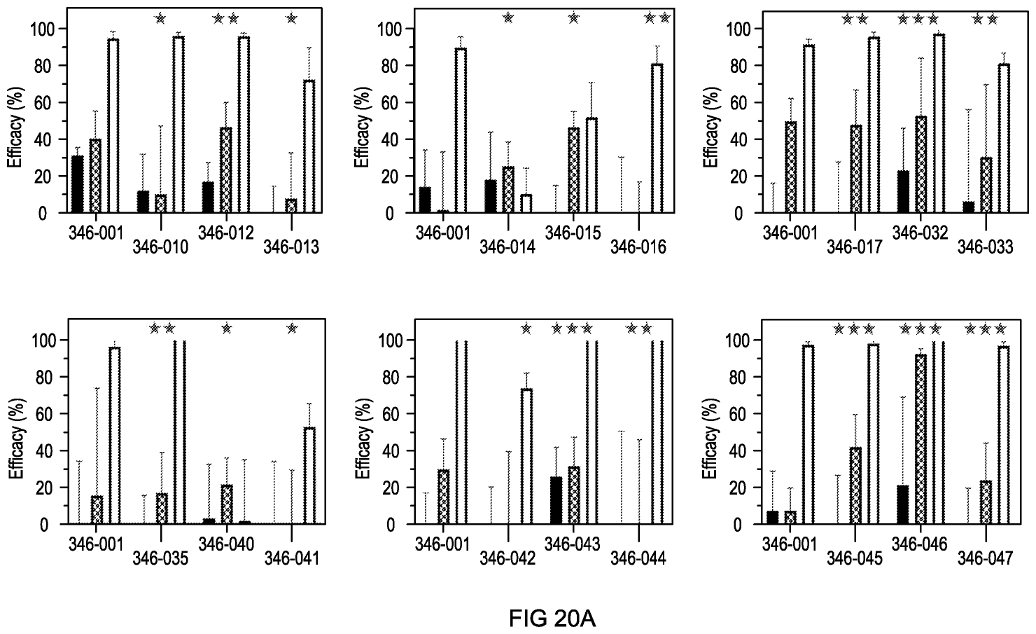

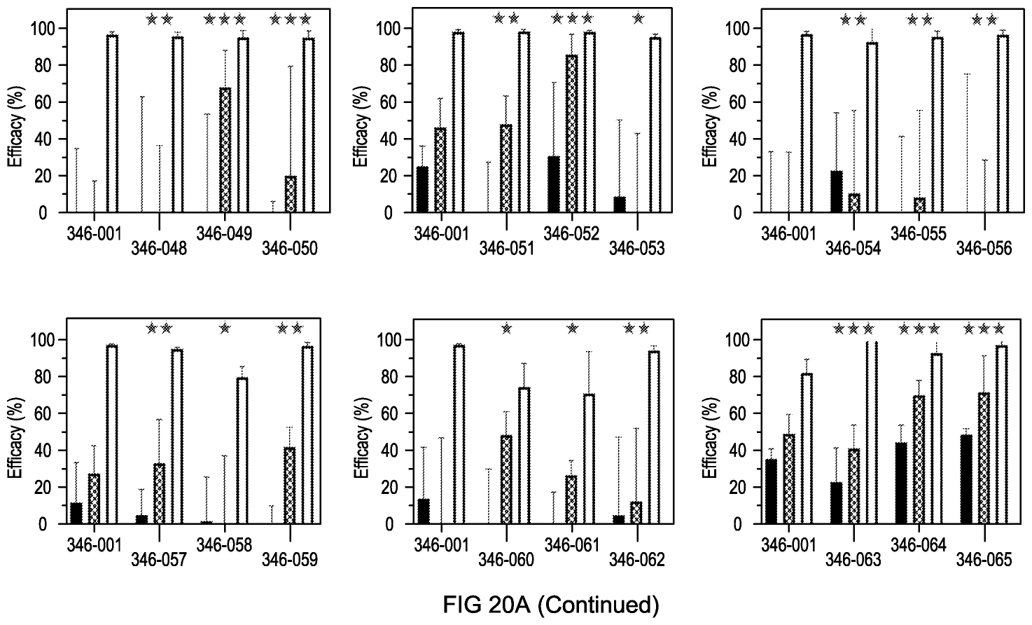

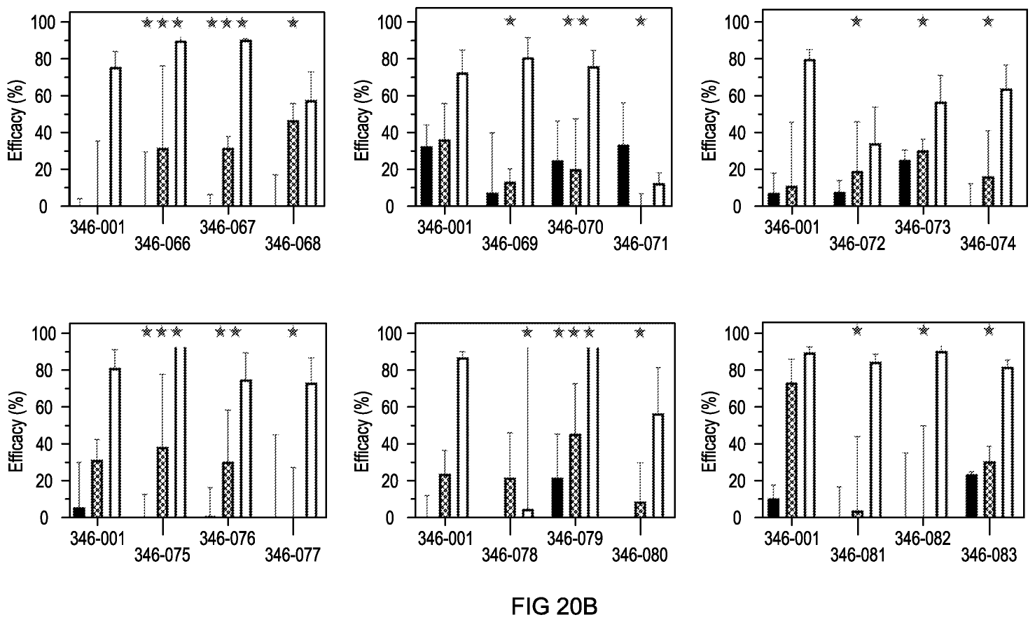

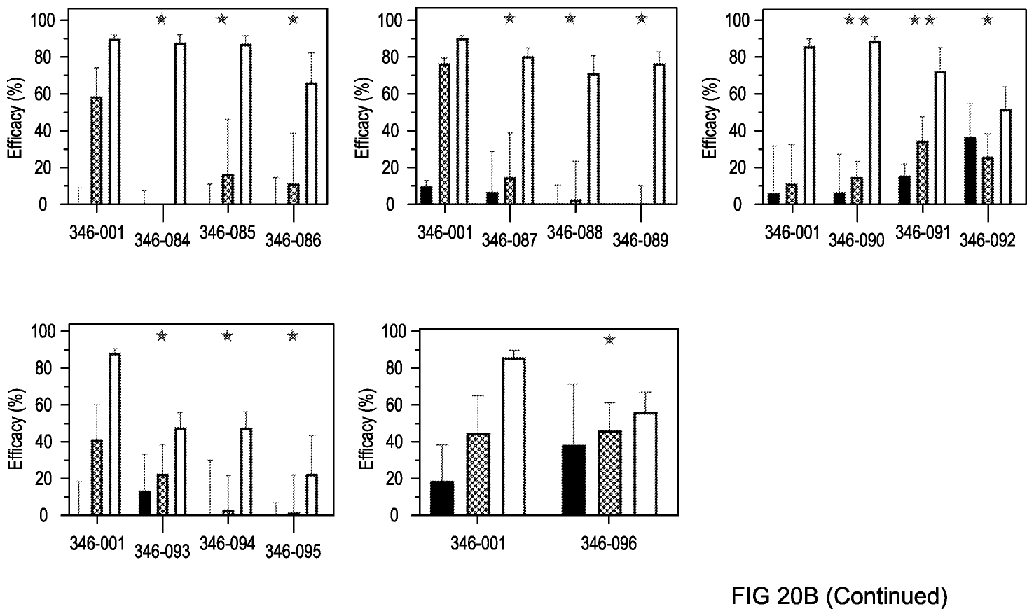

The study was carried out using a 96-well format. The first row of 12 wells was used as a negative control (cells with no virus or test compound added). The last row of 12 wells was used as a negative control (cells with virus, but no test compound added). The remaining wells were used to evaluate four compounds (6 replicates, 3 concentrations, 6×3=18 wells per compound). Peptide 346-001 served as a positive control on each plate, such that three other test compounds could be evaluated per plate. This format is reflected in the layout of the FIG. 20A-B panels.

Results from the study are shown in FIGS. 20A-B. The primary conclusions from the above study include a number of surprising findings. The peptides demonstrated inhibitory activity against HIV. A number (18/67) of the peptides exhibited a potency against HIV-1 comparable to peptide 346-001, while a number (15/67, 22%) of peptides demonstrated a potency against HIV-1 greater than peptide 346-001. Interestingly, modifications at positions 1, 2, and 3 (starting from the N-terminus) of the amino acid sequence peptide 346-001 resulted in a dramatic reduction in potency against HIV-1. Modifications at positions 4, 5, 6, 7, 10, 13, and 18 (starting from the N-terminus) in reference to peptide 346-001 resulted in an increase in potency against HIV-1. Modifications to at position 11 to substitute the cysteine residue with a different amino acid had variable effects against HIV-1: Cys to Ser did not affect potency; Cys to Ala increased potency; Cys to lie reduced potency; Cys to Trp reduced potency; Cys to Arg did not affect potency; and Cys to Pro reduced potency; Cys to Val reduced potency. Lipoconjugation of peptide 346-001 at the N- or C-termini did not result in an increase in potency, except C-terminal conjugation with Cys(succinimido-propionylaminoethyl-mPEG2K)-NH2 (peptide 346-064). When single modifications to peptide 346-001 that increased potency against HIV-1 were combined, the increase in potency against HIV-1 generally was not additive. Additionally, capping the N- and C-termini as amides did not result in a change in potency.

TABLE 1
Examples of peptides against viruses.

| | SEQ ID | |
|---|---|---|
| Peptide | NO: | Peptide Sequence and Modifications |
| 346-001 | 3 | SWLRDIWDWICEVLSDFK |
| 346-002 | 4 | swlrdiwdwicevlsdfk |
| 346-003 | 5 | GSWLRDIWDWICEVLSDFK |
| 346-004 | 6 | SWLRDIWDWICEVLSDFKTW |
| 346-005 | 7 | kfdslveciwdwidrlws |
| 346-006 | 8 | KWLCRIWSWISDVLDDFE |
| 346-007 | 9 | SIWRDWVDLICEFLSDWK |
| 346-008 | 10 | DWLRIIWDWVCSVVSDFK |
| 346-010 | 11 | SWLRDIWDWISEVLSDFK |
| 346-011 | 12 | swlrdiwdwisevlsdfk |
| 346-012 | 13 | SWLRDIWDWICEVLSDFR |
| 346-013 | 14 | SYLRDIWDYICEVLSDFK |
| 346-014 | 15 | SYLRDIWDYISEVLSDFK |
| 346-015 | 16 | SYLREIWDYISEVLSDFR |
| 346-016 | 17 | SWLREIWDWICEVLSDFK |
| 346-017 | 18 | Ac-SWLRDIWDWICEVLSDFK-NH₂ |
| 346-018 | 19 | Ac-swlrdiwdwicevlsdfk-NH₂ |
| 346-019 | 20 | Ac-GSWLRDIWDWICEVLSDFK-NH₂ |
| 346-020 | 21 | Ac-SWLRDIWDWICEVLSDFKTW-NH₂ |
| 346-021 | 22 | Ac-kfdslveciwdwidrlws-NH₂ |
| 346-022 | 23 | Ac-KWLCRIWSWISDVLDDFE-NH₂ |
| 346-023 | 24 | Ac-SIWRDWVDLICEFLSDWK-NH₂ |
| 346-024 | 25 | Ac-DWLRIIWDWVCSVVSDFK-NH₂ |
| 346-025 | 26 | Ac-SWLRDIWDWISEVLSDFK-NH₂ |
| 346-026 | 27 | Ac-swlrdiwdwisevlsdfk-NH₂ |
| 346-027 | 28 | Ac-SWLRDIWDWICEVLSDFR-NH₂ |
| 346-028 | 29 | Ac-SYLRDIWDYICEVLSDFK-NH₂ |
| 346-029 | 30 | Ac-SYLRDIWDYISEVLSDFK-NH₂ |
| 346-030 | 31 | Ac-SYLREIWDYISEVLSDFR-NH₂ |
| 346-031 | 32 | Ac-SWLREIWDWICEVLSDFK-NH₂ |
| 346-032 | 33 | SWLRDIWDWISEVLSDFR |
| 346-033 | 34 | SWLRDIWDYISEVLSDFR |
| 346-034 | 35 | swlrdiwdyisevlsdfr |
| 346-035 | 36 | SWLRDIWDYICEVLSDFR |
| 346-036 | 37 | Ac-SWLRDIWDWISEVLSDFR-NH₂ |
| 346-037 | 38 | Ac-SWLRDIWDYISEVLSDFR-NH₂ |
| 346-038 | 39 | Ac-swlrdiwdyisevlsdfr-NH₂ |
| 346-039 | 40 | Ac-SWLRDIWDYICEVLSDFR-NH₂ |
| 346-040 | 41 | AWLRDIWDWICEVLSDFK |
| 346-041 | 42 | SALRDIWDWICEVLSDFK |
| 346-042 | 43 | SWARDIWDWICEVLSDFK |
| 346-043 | 44 | SWLADIWDWICEVLSDFK |
| 346-044 | 45 | SWLRAIWDWICEVLSDFK |
| 346-045 | 46 | SWLRDAWDWICEVLSDFK |
| 346-046 | 47 | SWLRDIADWICEVLSDFK |
| 346-047 | 48 | SWLRDIWAWICEVLSDFK |
| 346-048 | 49 | SWLRDIWDAICEVLSDFK |
| 346-049 | 50 | SWLRDIWDWACEVLSDFK |
| 346-050 | 51 | SWLRDIWDWIAEVLSDFK |
| 346-051 | 52 | SWLRDIWDWICAVLSDFK |
| 346-052 | 53 | SWLRDIWDWICEALSDFK |
| 346-053 | 54 | SWLRDIWDWICEVASDFK |
| 346-054 | 55 | SWLRDIWDWICEVLADFK |
| 346-055 | 56 | SWLRDIWDWICEVLSAFK |
| 346-056 | 57 | SWLRDIWDWICEVLSDAK |
| 346-057 | 58 | SWLRDIWDWICEVLSDFA |
| 346-058 | 59 | Ac-SWLRDIWDWICEVLSDFKK |
| 346-059 | 60 | KSWLRDIWDWICEVLSDFK-NH₂ |
| 346-060 | 61 | Ac-K(palmitoyl)-SWLRDIWDWICEVLSDFK-NH₂ |
| 346-061 | 62 | Ac-SWLRDIWDWICEVLSDFK-K(palmitoyl)-NH₂ |
| 346-062 | 63 | Ac-SWLRDIWDWICEVLSDFK-amino-PEG12-propionyl-NH₂ |
| 346-063 | 64 | Ac-amino-PEG12-propionyl-SWLRDIWDWICEVLSDFK-NH₂ |
| 346-064 | 65 | Ac-SWLRDIWDWICEVLSDFKC(succinimido- |
| | | propionylaminoethyl-mPEG2K)-NH₂ |
| 346-065 | 66 | SWLRSIWDWICEVLSDFK |
| 346-066 | 67 | SWLRLIWDWICEVLSDFK |
| 346-067 | 68 | SWLRFIWDWICEVLSDFK |
| 346-068 | 69 | SWLRDIWDWIIEVLSDFK |
| 346-069 | 70 | SWLRDIWDWIWEVLSDFK |
| 346-070 | 71 | SWLRDIWDWIREVLSDFK |
| 346-071 | 72 | SWLRDIWDWIPEVLSDFK |
| 346-072 | 73 | SWLRDIWDWIR |
| 346-073 | 74 | SIWRDIWDLAVEAVSDWK |
| 346-074 | 75 | SILRDIWDWICEVLSDFK |
| 346-075 | 76 | SLWRDIWDWICEVLSDFK |
| 346-076 | 77 | SWLRDIWDLICEVLSDFK |
| 346-077 | 78 | SWLRDIWDWIVEVLSDFK |
| 346-078 | 79 | SWLRDIWDWICEAVSDFK |
| 346-079 | 80 | SWLRDIWDWICEVLSDWK |
| 346-080 | 81 | SWLQRIWQWISDVLSEFE |
| 346-081 | 82 | SWLQDIWDWICEVLSDFK |
| 346-082 | 83 | SWLRRIWDWICEVLSDFK |
| 346-083 | 84 | SWLRDIWQWICEVLSDFK |
| 346-084 | 85 | SWLRDIWDWICDVLSDFK |
| 346-085 | 86 | SWLRDIWDWICEVLSEFK |
| 346-086 | 87 | SWLRDIWDWICEVLSDFE |
| 346-087 | 88 | SWLRAIWDWISEVLSDFR |
| 346-088 | 89 | SWLRaIWDWISEVLSDFR |
| 346-089 | 90 | SWLRAIWDWIsEVLSDFR |
| 346-090 | 91 | SWLRAIWDWISEVLSDFr |
| 346-091 | 92 | Ac-SWLRDIWDWICEVLSDFK-K(oleoyl)-NH₂ |
| 346-092 | 93 | Ac-K(succinyl-D-a-tocopherol)-SWLRDIWDWICEVLSDFK-NH₂ |
| 346-093 | 94 | Ac-SWLRDIWDWICEVLSDFK-K(succinyl-D-a-tocopherol)-NH₂ |
| 346-094 | 95 | SWLRDIWDWICEVLSDFK-K(palmitoyl)-NH₂ |
| 346-095 | 96 | Ac-C(succinimido-propionylaminoethyl-mPEG2K)- |
| | | SWLRDIWDWICEVLSDFK-NH₂ |
| 346-096 | 97 | SWLRDIWDWICEVLSDFK-C(cholesteryloxycarbonylmethyl)-NH₂ |
| 346-097 | 98 | SWLRDIADWIAEVLSDFA |
| 346-098 | 99 | SWLRAIADWIAEVLSDFK |
| 346-099 | 100 | Ac-SWLRDIADWIAEVLSDFA-NH2 |
| 346-100 | 101 | Ac-SWLRAIADWIAEVLSDFK-NH2 |
| 346-101 | 102 | Ac-SWLRDIADWICEVLSDFK-NH2 |
| 346-102 | 103 | Ac-SWLRAIWDWISEVLSDFR-NH2 |
| 346-103 | 104 | SWLRDIWDWIGEVLSDFK |
| 346-104 | 105 | SWLRDIADWIAEVLSDFK |
| 346-105 | 106 | SWLRDIGDWIAEVLSDFK |
| 346-106 | 107 | SWLRDIADWIAEVLSDFR |
| 346-107 | 108 | SWLRDIADWICEVLSDWK |
| 346-108 | 109 | SWLRDIADWICEALSDFK |

Upper case single letter amino acid abbreviations indicate L-isomer; lower case single letter amino acid abbreviations indicate D-isomer.

TABLE 2
Examples of general peptide conjugates based on Peptide 346-001 backbone.

Conj-Ser-Trp-Leu-Arg-Asp-Ile-Trp-Asp-Trp-Ile-Cys-Glu-Val-Leu-Ser-Asp-Phe-Lys-NH₂
(SEQ ID NO: 110)
Ac-Ser-Trp-Leu-Arg-Asp-Ile-Trp-Asp-Trp-Ile-Cys-Glu-Val-Leu-Ser-Asp-Phe-Lys-Conj
(SEQ ID NO: 111)
Conj-Ser-Trp-Leu-Arg-Asp-Ile-Trp-Asp-Trp-Ile-Cys-Glu-Val-Leu-Ser-Asp-Phe-Lys-Conj
(SEQ ID NO: 112)
Conj, denotes conjugate

All references cited herein are incorporated by reference in their entirety as though fully set forth herein. Unless defined otherwise, technical and scientific terms used herein have the same meaning as commonly understood by one of ordinary skill in the art. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (Boca Raton, FL, 2008); Oxford Textbook of Medicine, Oxford Univ. Press (Oxford, England, UK, May 2010, with 2018 update); Harrison's Principles of Internal Medicine, Vol. 1 and 2, 20^{th} ed., McGraw-Hill (New York, NY, 2018); Singleton and Sainsbury, Dictionary of Microbiology and Molecular Biology, 3^{rd} ed., revised ed., J. Wiley & Sons (New York, NY, 2006); Smith, March's Advanced Organic Chemistry Reactions, Mechanisms and Structure 7^{th} ed., J. Wiley & Sons (New York, NY, 2013); and Singleton, Dictionary of DNA and Genome Technology, 3^{rd} ed., Wiley-Blackwell (Hoboken, NJ, 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. The entire document is intended to be related as a unified disclosure, and it should be understood that all combinations of features described herein (even if described in separate sections) are contemplated, even if the combination of features is not found together in the same sentence, or paragraph, or section of this document.

It should be understood that, while various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” or “consist essentially of” the feature. The term “a” or “an” refers to one or more, and the terms “a” (or “an”), “one or more,” and “at least one” can be used interchangeably herein. The term “or” should be understood to encompass items in the alternative or together, unless context unambiguously requires otherwise.

### REFERENCES CITED

1. UNAIDS Prevailing against Pandemics by Putting People at the Centre—World AIDS Day Report 2020; UNAIDS/JC3007E; UNAIDS: Geneva, CH, 2020; 92 pages.
2. UNAIDS Global Factsheet 2017. AIDSinfo.unaids.org (accessed April 15).
3. Bobardt, M. D. et al. Proc. Natl. Acad. Sci. U.S.A. 2008, 105 (14), 5525-5530.
4. Cheng, G. et al. Proc. Natl. Acad. Sci. U.S.A. 2008, 105 (8), 3088-3093.
5. Kolb, H. C. et al. Angew. Chem. Int. Ed. Engl. 2001, 40 (11), 2004-2021.
6. Kolb, H. C. et al. Drug Discov. Today 2003, 8 (24), 1128-1137.
7. Moses, J. E. et al. Chem. Soc. Rev. 2007, 36 (8), 1249-1262.
8. Meldal, M. et al. Chem. Rev. 2008, 108 (8), 2952-3015.
9. Bundgaard, H., J. Control. Release 1992, 21 (1-3), 63-72.
10. Oliyai, R. et al. Annu. Rev. Pharmacol. Toxicol. 1993, 33, 521-544.
11. Madani, N. et al. Nat. Commun. 2018, 9 (1), 2363.
12. Alexandre, K. B. et al. Virology 2012, 423 (2), 175-186.
13. Teh, A. Y. H. et al. Plant Biotechnol. J. 2014, 12 (3), 300-311.
14. Shaughnessy, J. et al. J. Immunol. 2018, 201 (9), 2700-2709.
15. Chakraborti, S. et al. Infect. Immun. 2020, 88 (2).
16. Gulati, S. et al. J. Immunol. 2020, 204 (12), 3283-3295.
17. Shaughnessy, J. et al. Front. Immunol. 2020, 11, 583305.
18. Wang, Y. et al. Adv. Drug Deliv. Rev. 2017, 110-111, 112-126.
19. Young Holt, B. et al. Biol. Reprod. 2020, 103 (2), 286-288.
20. Friend, D. R. et al. Antiviral Res. 2010, 88, S47-S54.
21. Friend, D. R. et al. Expert Opin. Drug Deliv. 2012, 9 (4), 417-427.
22. Friend, D. R. et al. Expert Opin. Drug Deliv. 2016, 13 (4), 533-545.
23. Quaife, M. et al. Health Econ. 2016, 25, 116-123.
25. Brady, M. et al. Antiviral Res. 2013, 100, S25-S31.
26. Boonstra, H. et al. B.J.O.G. 2014, 121, 23-26.
27. Woodsong, C. et al. Adv. Drug Deliv. Rev. 2015, 92, 146-154.
28. Hynes, J. S. et al. Contraception 2018, 97 (3), 277-284.
29. Minnis, A. M. et al. J. Acquir. Immune Defic. Syndr. 2019, 80 (4), 394-403.
30. Boyd, P. et al. Int. J. Pharm. 2016, 511 (1), 619-629.
31. Clark, J. T. et al. PLoS One 2014, 9 (3).
32. Achilles, S, et al. HIV R4P, Jan. 28, 2021; p OA06.01.
33. Mugo, N, et al. HIV R4P, Jan. 28, 2021; p OA06.02.
34. Thurman, A, et al. HIV R4P, Jan. 28, 2021; p OA06.03.
35. Westhoff, C. L. et al. Am. J. Obstet. Gynecol. 2007, 196 (4), e1-6.
36. Polis, C. B. et al. AIDS 2016, 30 (17), 2665-2683.
37. Ray, R. M. et al. Sci. Rep. 2019, 9.
38. Polis, C. B. et al. AIDS 2016, 30 (17), 2571-2576.
39. Seidman, D. et al. Semin. Reprod. Med. 2016, 34 (3), 159-167.
40. Bosinger, S. E. et al. Am. J. Reprod. Immunol. 2018, 80 (4).
41. Dupont, H. A. et al. Am. J. Reprod. Immunol. 2018, 80 (2).
42. Thurman, A. R. et al. AIDS Res. Hum. Retroviruses 2019, Epub ahead of print April 18.
43. Saxena, B. B. et al. Contraception 2004, 70 (3), 213-219.
44. Han, Y. A. et al. Contraception 2007, 76 (2), 132-138.
45. Balbach, M. et al. Biol. Reprod. 2020, 103 (2), 176-182.
46. O'Rand, M. G. et al. Pharmacol. Ther. 2016, 157, 105-111.
47. O'Rand, M. G. et al. PLoS One 2018, 13 (4), e0195953.
48. Faber, E. B. et al. Biol. Reprod. 2020, 103 (2), 357-367.
49. Anderson, D. J. et al. Biol. Reprod. 2020, 103 (2), 275-285.
50. Shrestha, B. et al. Acta Biomater. 2020, 117, 226-234.
51. Chang, L. et al. J. Pharm. Sci. 2009, 98 (9), 2886-2908.
52. Mensink, M. A. et al. Eur. J. Pharm. Biopharm. 2017, 114, 288-295.
53. Forney-Stevens, K. M. et al. J. Pharm. Sci. 2015, 105, 697-704.
54. Li J. et al. Pharmaceutics. 2019; 12(1):1.
55. Gong T. et al. J Pharm Innov. 2017; 12(2):142-154.
56. Zwart G. et al. AIDS Res Hum Retroviruses. 1992; 8(11):1897-1908.
57. Zanotto C. et al. Virology. 1995; 206(2):807-816.
58. Peraro L. et al. Angew Chem Int Ed Engl. 2018; 57(37):11868-11881.
59. Apostolopoulos V. et al. Molecules. 2021; 26(2):430.

What is claimed is:

1. A method of treating or preventing a viral non-respiratory disease, the method comprising administering to a subject in need thereof a peptide comprising
(Formula 1; SEQ ID NO: 1)
X₁-X₂-X₃-X₄-X₅-X₆-X₇-X₈-X₉-X₁₀-X₁₁-X₁₂-X₁₃-X₁₄-X₁₅-X₁₆-X₁₇-
X₁₈,
wherein
X₁ is S, K, D, A, N, M, T, or V; X₂ is W, I, Y, F, P, L, V, H, M, A, T, or S; X₃ is L, W, I, M, V, F, M, A, or T; X₄ is R, C, K, Q, H, P, or C; X₅ is D, R, I, E, N, or Y; X₆ is I, W, V, L, or F; X₇ is W, V, Y, F, P, L, V, H, or D; X₈ is D, E, N, Y, or S; X₉ is W, L, Y, F, P, L, V, or H; X₁₀ is I, V, L, or F; X₁₁ is C, S, A, P, M, H, or T; X₁₂ is E, D, S, Q, Y, or T; X₁₃ is V, F, I, L, A, or M; X₁₄ is L, V, I, M, or F; X₁₅ is S, D, A, N, T, Y, or P; X₁₆ is D, E, N, Y, or S; X₁₇ is F, W, Y, L, P, or H; and X₁₈ is K, E, H, R, P, or Q.

2. The method of claim 1, wherein the peptide comprises the amino acid sequence of Peptide 346-001.

3. The method of claim 1, wherein the peptide comprises the amino acid sequence of Peptide 346-001 having one, two, or three amino acid substitutions, the substitutions being selected from the following: the S at position 1 is substituted with K, D, A, N, M, T, or V; the W at position 2 is substituted with I, Y, F, P, L, V, H, M, A, T, or S; the L at position 3 is substituted with W, I, M, V, F, M, A, or T; the R at position 4 is substituted with C, K, Q, H, P, or C; the D at position 5 is substituted with R, I, E, N, or Y; the I at position 6 is substituted with W, V, L, or F; the W at position 7 is substituted with V, Y, F, P, L, V, H, or D; the D at position 8 is substituted with E, N, Y, or S; the W at position 9 is substituted with L, Y, F, P, L, V, or H; the I at position 10 is substituted with V, L, or F; the C at position 11 is substituted with S, A, P, M, H, or T; the E at position 12 is substituted with D, S, Q, Y, or T; the V at position 13 is substituted with F, I, L, A, or M; the L at position 14 is substituted with V, I, M, or F; the S at position 15 is substituted with D, A, N, T, Y, or P; the D at position 16 is substituted with E, N, Y, or S; the F at position 17 is substituted with W, Y, L, P, or H; or the K at position 18 is substituted with E, H, R, P, or Q.

4. The method of claim 3, wherein the substitutions are selected from the following: the D at position 5 is substituted with I, E, or Y; the I at position 6 is substituted with W, V, L, or F; the W at position 7 is substituted with V, F, or L; the D at position 8 is substituted with E, Y, or S; the I at position 10 is substituted with V, L, or F; the C at position 11 is substituted with S, A, M, H, or T; the V at position 13 is substituted with F, I, L, A, or M; the F at position 17 is substituted with W, Y, L, or H; or the K at position 18 is substituted with H, R, or P.

5. The method of any one of claims 1 to 4, wherein X₁ is S; X₂ is W; X₃ is L; X₉ is W; X₁₂ is E; X₁₄ is L; and X₁₅ is S,

6. The method of any one of claims 1 to 5, wherein the peptide is modified with a lipophilic moiety via a linker bonded to a lysine, serine, aspartic acid, glutamic acid, or cysteine of the peptide or variant.

7. The method of any one of claims 1 to 6, wherein the viral non-respiratory disease is an HIV infection.

8. The method of claim 7, wherein the viral non-respiratory disease is an HIV-1 or HIV-2 infection.

9. The method of any one of claims 1 to 6, wherein the viral non-respiratory disease is an HSV infection.

10. The method of claim 9, wherein the viral non-respiratory disease is an HSV-1 or HSV-2 infection.

11. The method of any one of claims 1 to 10, further comprising administering one or more complementary agents.

12. The method of claim 11, wherein the one or more complementary agents comprises a contraceptive.

13. The method of claim 12, wherein the contraceptive is a non-hormonal contraceptive.

14. The method of any one of claims 1 to 13, wherein the peptide comprises an amino acid sequence of peptides 346-002 through 342-064 set forth in TABLE 1.

15. The method of any one of claims 1 to 14, wherein X₁₁ is not C.

16. The method of any one of claims 1 to 15, wherein one or more amino acids are D-amino acids.

17. A method of treating or preventing a viral non-respiratory disease, the method comprising administering to a subject in need thereof a peptide comprising
S-W-L-X₄-X₅-X₆-X₇-X₈-W-X₁₀-X₁₁-E-X₁₃-L-S-D-X₁₇-X₁₈ (Formula 2; SEQ ID NO: 2), wherein X₄ is R, G, A, I, L, V, or F; X₅ is D, G, I, L, V, F, S, T, E, or Y; X₆ is I, A, W, V, L, or F; X₇ is W, A, V, Y, F, L, V, H, or D; X₈ is D, A, S, E, or Y; X₁₀ is I, A, V, L, or F; X₁₁ is C, S, A, W, M, H, W, R, V, or T; X₁₃ is V, F, I, L, A, or M; X₁₇ is F, W, Y, L, or H; and X₁₈ is K, R, H, A, P, or G.

18. The method of claim 17, wherein the peptide comprises the amino acid sequence of peptide 346-001 having an amino acid substitution at four amino acid positions selected from positions X₄, X₅, X₆, X₇, X₈, X₁₀, X₁₁, X₁₃, X₁₇, and X₁₈.

19. The method of claim 17, wherein the peptide comprises the amino acid sequence of peptide 346-001 having an amino acid substitution at three amino acid positions selected from positions X₄, X₅, X₆, X₇, X₈, X₁₀, X₁₁, X₁₃, X₁₇, and X₁₈.

20. The method of claim 17, wherein the peptide comprises the amino acid sequence of peptide 346-001 having an amino acid substitution at two amino acid positions selected from positions X₄, X₅, X₆, X₇, X₈, X₁₀, X₁₁, X₁₃, X₁₇, and X₁₈.

21. The method of claim 17, wherein the peptide comprises the amino acid sequence of peptide 346-001 having an amino acid substitution at one amino acid position selected from positions X₄, X₅, X₆, X₇, X₈, X₁₀, X₁₁, X₁₃, X₁₇, and X₁₈.

22. The method of any one of claims 18 to 21, wherein at least one of the amino acid substitutions is at position X₄, X₅, or X₆.

23. The method of any one of claims 18 to 21, wherein at least one of the amino acid substitutions is at position X₇, X₈, or X₁₀.

24. The method of any one of claims 18 to 23, wherein at least one of the amino acid substitutions is at position X₁₃, X₁₇, or X₁₈.

25. The method of any one of claims 18 to 24, wherein the peptide comprises an amino acid substitution at position X₁₁.

26. The method of any one of claims 17 to 25, wherein if X₄ is not R, it is G, A, I, L, V, or F.

27. The method of any one of claims 17 to 26, wherein if X₅ is not D, it is G, I, L, V, F, S, T, E, or Y; optionally I, E, or Y.

28. The method of any one of claims 17 to 27, wherein if X₆ is not I, it is A, W, V, L, or F;
optionally W, V, L, or F.

29. The method of any one of claims 17 to 28, wherein if X₇ is not W, it is A, V, Y, F, L, V, H, or D; optionally V, Y, F, L, V, H, or D.

30. The method of any one of claims 17 to 29, wherein if X₈ is not D, it is A, S, E, or Y; optionally, E, Y, or S.

31. The method of any one of claims 17 to 30, wherein if X₁₀ is not I, it is A, V, L, or F; optionally V, L, or F.

32. The method of any one of claims 17 to 31, wherein if X₁₁ is not C, it is S, A, W, M, H, W, R, V, or T; optionally S, A, M, H, or T.

33. The method of any one of claims 17 to 32, wherein if X₁₃ is not V, it is F, I, L, A, or M.

34. The method of any one of claims 17 to 33, wherein if X₁₇ is not F, it is W, Y, L, or H.

35. The method of any one of claims 17 to 34, wherein if X₁₈ is not K, it is R, H, A, P, or G; optionally R, H, or P.

36. The method of any one of claims 17 to 35, wherein the viral non-respiratory disease is an HIV infection.

37. The method of claim 36, wherein the viral non-respiratory disease is and HIV-1 or HIV-2 infection.

38. The method of any one of claims 17 to 35, wherein the viral non-respiratory disease is an HSV infection.

39. The method of claim 38, wherein the viral non-respiratory disease is an HSV-1 or HSV-2 infection.

40. The method of any one of claims 17 to 39, further comprising administering one or more complementary agents.

41. The method of claim 40, wherein the one or more complementary agents comprise a contraceptive.

42. The method of claim 41, wherein the contraceptive is a non-hormonal contraceptive.

43. The method of any one of claims 17 to 42, wherein one or more amino acids are D-amino acids.

44. A composition comprising a peptide comprising the sequence:
S-W-L-X₄-X₅-X₆-X₇-X₈-W-X₁₀-X₁₁-E-X₁₃-L-S-D-X₁₇-X₁₈ (Formula 2; SEQ ID NO: 2), wherein X₄ is R, G, A, I, L, V, or F; X₅ is D, G, I, L, V, F, S, T, E, or Y; X₆ is I, A, W, V, L, or F; X₇ is W, A, V, Y, F, L, V, H, or D; X₈ is D, A, S, E, or Y; X₁₀ is I, A, V, L, or F; X₁₁ is C, S, A, W, M, H, W, R, V, or T; X₁₃ is V, F, I, L, A, or M; X₁₇ is F, W, Y, L, or H; and X₁₈ is K, R, H, A, P, or G, wherein the peptide is not peptide 346-001.

45. The composition of claim 44, wherein the peptide comprises the amino acid sequence of peptide 346-001 having an amino acid substitution at four amino acid positions selected from positions X₄, X₅, X₆, X₇, X₈, X₁₀, X₁₁, X₁₃, X₁₇, and X₁₈.

46. The composition of claim 44, wherein the peptide comprises the amino acid sequence of peptide 346-001 having an amino acid substitution at three amino acid positions selected from positions X₄, X₅, X₆, X₇, X₈, X₁₀, X₁₁, X₁₃, X₁₇, and X₁₈.

47. The composition of claim 44, wherein the peptide comprises the amino acid sequence of peptide 346-001 having an amino acid substitution at two amino acid positions selected from positions X₄, X₅, X₆, X₇, X₈, X₁₀, X₁₁, X₁₃, X₁₇, and X₁₈.

48. The composition of claim 44, wherein the peptide comprises the amino acid sequence of peptide 346-001 having an amino acid substitution at one amino acid position selected from positions X₄, X₅, X₆, X₇, X₈, X₁₀, X₁₁, X₁₃, X₁₇, and X₁₈.

49. The composition of any one of claims 45 to 48, wherein at least one of the amino acid substitutions is at position X₄, X₅, or X₆.

50. The composition of any one of claims 45 to 48, wherein at least one of the amino acid substitutions is at position X₇, X₈, or X₁₀.

51. The composition of any one of claims 45 to 50, wherein at least one of the amino acid composition is at position X₁₃, X₁₇, or X₁₈.

52. The method of any one of claims 45 to 51, wherein the peptide comprises an amino acid substitution at position X₁₁.

53. The composition of any one of claims 44 to 52, wherein if X₄ is not R, it is G, A, I, L, V, or F.

54. The composition of any one of claims 44 to 53, wherein if X₅ is not D, it is G, I, L, V, F, S, T, E, or Y; optionally I, E, or Y.

55. The composition of any one of claims 44 to 54, wherein if X₆ is not I, it is A, W, V, L, or F; optionally W, V, L, or F.

56. The composition of any one of claims 44 to 55, wherein if X₇ is not W, it is A, V, Y, F, L, V, H, or D; optionally V, Y, F, L, V, H, or D.

57. The composition of any one of claims 44 to 56, wherein if X₈ is not D, it is A, S, E, or Y; optionally, E, Y, or S.

58. The composition of any one of claims 44 to 57, wherein if X₁₀ is not I, it is A, V, L, or F; optionally V, L, or F.

59. The composition of any one of claims 44 to 58, wherein if X₁₁ is not C, it is S, A, W, M, H, W, R, V, or T; optionally S, A, M, H, or T.

60. The composition of any one of claims 44 to 59, wherein if X₁₃ is not V, it is F, I, L, A, or M.

61. The composition of any one of claims 44 to 60, wherein if X₁₇ is not F, it is W, Y, L, or H.

62. The composition of any one of claims 44 to 61, wherein if X₁₈ is not K, it is R, H, A, P, or G; optionally R, H, or P.

63. The composition of any one of claims 44 to 62, wherein one or more amino acids are D-amino acids.
